(19) United States
(12) Patent Application Publication
(10) Pub. No.: US 2024/0011983 A1
(43) Pub. Date: Jan. 11, 2024

(54) METHOD FOR DETERMINING WHETHER A SYSTEMIC LUPUS ERYTHEMATOSUS (SLE) PATIENT IS UNDERGOING A PRE-FLARE EVENT

(71) Applicants: Progentec Diagnostics, Inc., Oklahoma City, OK (US); Oklahoma Medical Research Foundation, Oaklahoma City, OK (US)

(72) Inventors: Melissa MUNROE, Oklahoma City, OK (US); Judith JAMES, Oklahoma City, OK (US); Eldon JUPE, Oklahoma City, OK (US); Mohan PURUSHOTHAMAN, Oklahoma City, OK (US)

(73) Assignees: Progentec Diagnostics, Inc., Oklahoma City, OK (US); Oklahoma Medical Research Foundation, Oaklahoma City, OK (US)

(21) Appl. No.: 18/016,384

(22) PCT Filed: Jul. 21, 2021
(86) PCT No.: PCT/US2021/042629

§ 371 Date: Jan. 16, 2023

Related U.S. Application Data
(60) Provisional application No. 63/055,251, filed on Jul. 22, 2020.

(51) Int. Cl. G01N 33/564

(52) U.S. Cl. CPC G01N 33/564; G01N 2333/96494; G01N 2333/54; G01N 2333/525; G01N 2800/104

(57) ABSTRACT

A method for determining whether a Systemic lupus erythematosus (SLE) patient is undergoing a pre-flare event, the method comprising obtaining a blood, serum, plasma, or saliva sample from the SLE patient; assessing a level of expression for each of a plurality of biomarkers, the plurality of biomarkers comprising OPN, MCP-1/CCL2, MCP-3/CCL7, IL-17A, TNFRII, TNFRI, IL-4, IL-5, BLyS, TNFα, and IL-7; determining a Lupus Flare Risk Prediction Index (LFPI) for the patient based upon the level of expression for each of a plurality of biomarkers; and based upon the LFPI, determining whether the patient is undergoing a pre-flare event.

### CROSS-REFERENCE TO RELATED APPLICATIONS

This application is a 371 U.S. National Phase of International Application No. PCT/US2021/042629, filed on Jul. 21, 2021, which claims priority to U.S. Provisional Application Ser. No. 63/055,251 filed Jul. 22, 2020 and entitled “Method for Determining Whether a Systemic Lupus Erythematosus (SLE) Patient is Undergoing a Pre-Flare Event” by Inventors Melissa Munroe, et al., which is incorporated here by reference in its entirety.

### TECHNICAL FIELD

The subject matter disclosed herein relates generally to the fields of autoimmune disease, immunology, rheumatology and molecular biology. More particularly, it concerns soluble inflammatory mediators that are predictive of and involved in systemic lupus erythematosus flares.

### BACKGROUND

Systemic lupus erythematosus (SLE) is a multifaceted autoimmune disease characterized by variable immune dysregulation, disabling symptoms and progressive organ damage. Given the heterogeneous nature of SLE, recognition and early treatment to prevent tissue and organ damage is clinically challenging. Validated disease activity clinical instruments assess and weight changes in signs and symptoms within each organ system. The Safety of Estrogens in Lupus Erythematosus National Assessment-Systemic Lupus Erythematosus Disease Activity Index (SELENA-SLEDAI) is a reliable measure of clinical disease activity. However, the traditional biomarkers incorporated in the SELENA-SLEDAI are not necessarily the earliest or sufficient biologic signals of worsening disease. Despite clinical instruments of disease activity and improved treatment regimens to temper chronic inflammation, SLE patients may experience an average of 1.8 disease flares annually. Treatment typically relies on rapidly acting, side effect-pervaded agents such as steroids. Earlier identification of flares might open the door for proactive strategies to reduce pathogenic and socioeconomic burdens of SLE. Further, uncovering early markers of clinical flares will provide mechanistic insight, improving the development of targeted preventative treatments. No serologic prognostic tool currently exists to identify SLE patients at risk of imminent disease flare; the lack of an immune mechanism-informed disease management test in SLE stems from no individual immune pathway-informed biomarker acting as a universal surrogate for flare risk.

Multiple inflammatory and regulatory mediators are known to be involved in SLE pathogenesis and disease flare, including innate and adaptive cytokines, chemokines, and altered regulation of soluble receptors expressed by activated immune cells. IL-6, TNF-α, and IL-10, as well as Th1 and Th2 type cytokines, have been implicated in SLE disease activity; elevated IL-12 has been detected prior to disease flare. Th17 pathway mediators have been implicated in increased disease activity and sequelae, including cutaneous, serositis, and renal manifestations. These changes, along with decreased TGF-β and reduced numbers of natural T-regulatory cells with active disease, suggest an imbalance between inflammatory and regulatory mediators in promoting flares.

Cytokines and chemokines are indicative of the ongoing immune response to (auto)antigens. In addition to soluble mediators of inflammation, SLE flares might also involve altered regulation of membrane-bound or soluble receptors expressed by activated cells. Members of the TNF-(R)eceptor superfamily form a prototypic pro-inflammatory system that act as co-stimulatory molecules on B and T-lymphocytes. The ligand/receptor pairings are either membrane bound or can be cleaved by proteases as soluble proteins that cluster as trimers to either block ligand/receptor interactions or to initiate receptor-mediated signal transduction. Multiple members of the TNF-R superfamily are implicated in SLE. The classical ligand TNF-α interacts with two TNFRs, TNFRI (p55) and TNFRII (p′75), both of which have been implicated in altered SLE disease activity. In addition, expression and cleavage of Fas, FasL, and CD40L/CD154 are increased in SLE patients. BLyS and APRIL, key regulators of B cell survival and differentiation, are important SLE therapeutic targets. In a study of 245 SLE patients followed for two years, with power to account for some confounding factors such as medications, increased BLyS levels associated with increased disease activity. Furthermore, a neutralizing anti-BLyS monoclonal antibody can reduce risk of disease flare over time, suggesting that BLyS may help regulate disease activity in some patients. However, their roles in ensuing disease flares are presently unknown.

### BRIEF SUMMARY

New and useful methods of determining whether a systemic lupus erythematosus (SLE) patient is undergoing a pre-flare event may comprise (a) obtaining a blood, serum, plasma, or saliva sample from a patient; (b) assessing the level of a biomarker; (c) determining a Lupus Flare Risk Prediction Index (LFPI) for the patient; and (d) based upon the LFPI, determining whether the patient is undergoing a pre-flare event.

The method may further comprise performing one or more of a SELENA-SLEDAI Index analysis on said patient, anti-dsDNA antibody (anti-dsDNA) testing in a sample from said patient and/or anti-extractable nuclear antigen (anti-ENA) in a sample from said patient. The method may further comprise taking a medical history of said patient. The method may further comprise treating said patient. The SLE patient not undergoing a flare event may be represented by a sample from the same patient during a non-flare period, or may be represented by a pre-determined average level.

In another embodiment, there is provided a method of assessing the efficacy of a treatment for systemic lupus erythematosus (SLE) in a patient comprising a) obtaining a blood, serum, plasma, or saliva sample from a patient; (b) assessing the level of a biomarker; (c) determining a Lupus Flare Risk Prediction Index (LFPI) for the patient; and (d) based upon the LFPI, determining whether the patient is undergoing a pre-flare event.

The method may further comprise performing one or more of a SELENA-SLEDAI Index analysis on said patient, anti-dsDNA antibody (anti-dsDNA) testing in a sample from said patient and/or anti-extractable nuclear antigen (anti-ENA) in a sample from said patient. The method may further comprise taking a medical history of said patient. The SLE patient not undergoing a flare event may be represented by a sample from the same patient during a non-flare period, or may be represented by a pre-determined average level.

Also provided is a kit comprising (a) one or more reagents for assessing the level of at least one of each of the following: innate type cytokine, Th1 type cytokine, Th2 type cytokine, and Th17 type cytokine, plus at least two each of the following: a chemokines/adhesion molecule, a TNFR superfamily member, a regulatory mediator, and other mediator previously shown to play a role in SLE pathogenesis; and (b) one or more reagents for assessing anti-dsDNA antibody (anti-dsDNA) testing and/or anti-extractable nuclear antigen (anti-ENA) in a biological sample.

The innate type cytokines may be selected from IL-1α, IL-1β, IFN-α, IFN-β, G-CSF, IL-7, and IL-15. The Th 1 type cytokine may be selected from IL-2, IL-12 and IFN-γ. The Th2 type cytokine may be selected from IL-4, IL-5 and IL-13. The Th17 type cytokine may be selected from IL-6, IL-17A, IL-21 and IL-23. Chemokines/adhesion molecules may be selected from IL-8, IP-10, RANTES, MCP-1, MCP-3, MIP-1α, MIP-1β, GRO-α, MIG, Eotaxin, ICAM-1, and E-selectin. TNFR superfamily members may be selected from TNF-α, TNFRI, TNFRII, TRAIL, Fas, FasL, BLyS, APRIL, and NGFβ. Other mediators previously shown to play a role in SLE pathogenesis may be selected from LIF, PAI-1, PDGF-BB, Leptin, SCF, osteopontin (OPN), and IL-2Rα. Regulatory mediators may be selected from IL-10, TGF-β, SDF-1 and IL-1RA. The reagents may be beads attached to binding ligands for each of said biomarkers, or ELISA based capture in microwell plates or microfluidic nanochambers.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1, shows performance results for the Lupus Flare Prediction Index (LFPI) informed by a subset of 11 mediators, termed group 11G. The calculated 11G LFPI significantly distinguishes lupus patients in a pre-flare state (positive risk score) vs. a pre-nonflare state (negative risk score).

FIG. 2 illustrates the effect of removing single immune mediators on performance of Flare Risk Index (FRI)-11, or “LFPI “11G” as described herein, as a box and whiskers (median±min/max IQR) differentiation of Pre-Flare vs. Pre-Nonflare samples of intact FRI-11 vs. FRI-11 minus single immune mediators in order of variable importance (****p<0.0001 Mann-Whitney test).

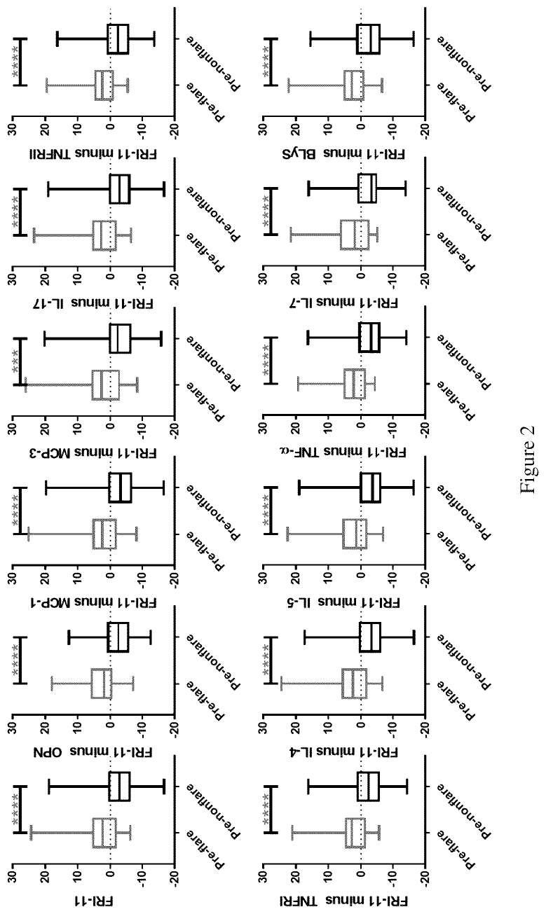

FIG. 3 illustrates the effect of removing single immune mediators on performance of Flare Risk Index (FRI)-11. Performance characteristics of FRI-11 ±removal of single immune mediators (AUC=Area Under the Curve [receiver operating characteristic curve analysis]), CI=confidence interval, PPV=positive predictive value, NPV=negative predictive value, Youden Index is point in ROC curve with maximum sensitivity and specificity). FRI-11 was selected for performance characteristics in combination with cost effectiveness for clinical/commercial application.

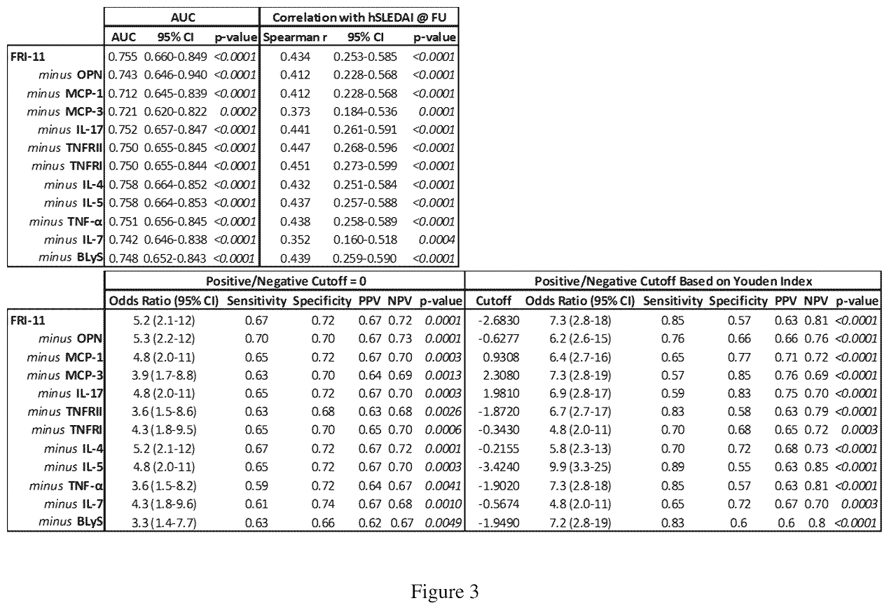

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

In some embodiments is a method for determining whether an SLE patient is undergoing a pre-flare event. In some embodiments, the method of determining whether the SLE patient is undergoing a pre-flare event may comprise (a) obtaining a blood, serum, plasma, or saliva sample from a patient; (b) assessing the level of a biomarker; (c) determining a Lupus Flare Risk Prediction Index (LFPI) for the patient; and (d) based upon the LFPI, determining whether the patient is undergoing a pre-flare event. In some embodiments, the methods disclosed herein may be effective to predict SLE flare, even before clinical symptoms are reported.

In some embodiments, the presently-disclosed methods present an optimized algorithm and capable of predicting SLE flares utilizing a reduced number of biomarkers required for adequate flare risk prediction, in comparison to prior methods. For example, the optimized algorithm for SLE flare prediction may utilize fewer than 20 biomarkers, for example, fewer that fifteen (15) biomarkers, for example, thirteen (13) biomarkers, for example, twelve (12) biomarkers for example, eleven (11) biomarkers. This advancement distinguishes the presently-disclosed subject matter from existing technology and represents an improvement upon earlier embodiments of the technology.

### Systemic Lupus Erythematosus (SLE)

### Disease Manifestations

SLE is a systemic autoimmune disease (or autoimmune connective tissue disease) that can affect any part of the body. The disease occurs nine times more often in women than in men, especially in women in child-bearing years ages 15 to 35, and is also more common in those of non-European descent.

As occurs in other autoimmune diseases, the immune system attacks the body's cells and tissue, resulting in inflammation and tissue damage. SLE can induce abnormalities in the adaptive and innate immune system, as well as mount Type III hypersensitivity reactions in which antibody-immune complexes precipitate and cause a further immune response. SLE most often damages the joints, skin, lungs, heart, blood components, blood vessels, kidneys, liver and nervous system. The course of the disease is unpredictable, often with periods of increased disease activity (called “flares”) alternating with suppressed or decreased disease activity. A flare has been defined as a measurable increase in disease activity in one or more organ systems involving new or worse clinical signs and symptoms and/or laboratory measurements. It must be considered clinically significant by the assessor and usually there would be at least consideration of a change or an increase in treatment.

SLE has no cure, and leads to increased morbidity and early mortality in many patients. The most common causes of death in lupus patients include accelerated cardiovascular disease (likely associated with increased inflammation and perhaps additionally increased by select lupus therapies), complications from renal involvement and infections. Survival for people with SLE in the United States, Canada, and Europe has risen to approximately 95% at five years, 90% at 10 years, and 78% at 20 years in patients of European descent; however, similar improvements in mortality rates in non-Caucasian patients are not as evident. Childhood systemic lupus erythematosus generally presents between the ages of 3 and 15, with girls outnumbering boys 4:1, and typical skin manifestations being butterfly eruption on the face and photosensitivity.

SLE is one of several diseases known as “the great imitators” because it often mimics or is mistaken for other illnesses. SLE is a classical item in differential diagnosis, because SLE symptoms vary widely and come and go unpredictably. Proper disease classification can thus be elusive, with some people suffering unexplained symptoms of untreated SLE for years. Common initial and chronic complaints include fever, malaise, joint pains, myalgias, fatigue, and temporary loss of cognitive abilities. Because they are so often seen with other diseases, these signs and symptoms may overlap across multiple American College of Rheumatology rheumatologic disease classification criteria. When occurring in conjunction with other signs and symptoms, however, they may be suggestive of SLE classification.

One common clinical symptom that may cause a patient to seek medical attention is joint pain; all joints are at risk. Between 80 and 90% of those affected will experience joint and/or muscle pain at some time during the course of their illness. Unlike rheumatoid arthritis, many lupus arthritis patients will have joint swelling and pain, but no X-ray changes and minimal loss of function. Fewer than 10% of people with lupus arthritis will develop deformities of the hands and feet. SLE patients are at particular risk of developing articular tuberculosis.

Over half (65%) of SLE patients have some dermatological manifestations at some point in their disease, with approximately 30% to 50% suffering from the classic malar rash (or butterfly rash) associated with the name of the disorder. Some may exhibit chronic thick, annual scaly patches on the skin (referred to as discoid lupus). Alopecia, mouth ulcers, nasal ulcers, and photosensitive lesions on the skin are also possible manifestations. Anemia may develop in up to 50% of lupus cases. In addition to SLE pathogenesis and ethnicity-associated hereditary neutropenia-leukopenia associated with minority populations, low platelet and white blood cell counts may be due to pharmacological treatment. People with SLE may have an association with antiphospholipid antibody syndrome (a thrombotic disorder), wherein autoantibodies to phospholipids are present in their serum. Abnormalities associated with antiphospholipid antibody syndrome include a paradoxical prolonged partial thromboplastin time (which usually occurs in hemorrhagic disorders) and a positive test for antiphospholipid antibodies; the combination of such findings has earned the term “lupus anticoagulant-positive.” SLE patients with anti-phospholipid autoantibodies have more ACR classification criteria of the disease and may suffer from a more severe lupus phenotype.

A person with SLE may have inflammation of various parts of the heart, such as pericarditis, myocarditis, and endocarditis. The endocarditis of SLE is characteristically non-infective (Libman-Sacks endocarditis), and involves either the mitral valve or the tricuspid valve. Atherosclerosis also tends to occur more often and advances more rapidly than in the general population. Lung and pleura inflammation can cause pleuritis, pleural effusion, lupus pneumonitis, chronic diffuse interstitial lung disease, pulmonary hypertension, pulmonary emboli, pulmonary hemorrhage, and shrinking lung syndrome.

Painless hematuria or proteinuria may often be the only presenting renal symptom. Acute or chronic renal impairment may develop with lupus nephritis, leading to acute or end-stage renal failure. Because of early recognition and management of SLE, end-stage renal failure occurs in less than 5% of cases. A histological hallmark of SLE is membranous glomerulonephritis with “wire loop” abnormalities. This finding is due to immune complex deposition along the glomerular basement membrane, leading to a typical granular appearance in immunofluorescence testing.

Neuropsychiatric syndromes can result when SLE affects the central or peripheral nervous systems. The American College of Rheumatology defines 19 neuropsychiatric syndromes in systemic lupus erythematosus. The diagnosis of neuropsychiatric syndromes concurrent with SLE is one of the most difficult challenges in medicine, because it can involve so many different patterns of symptoms, some of which may be mistaken for signs of infectious disease or stroke. The most common neuropsychiatric disorder people with SLE have is headache, although the existence of a specific lupus headache and the optimal approach to headache in SLE cases remains controversial. Other common neuropsychiatric manifestations of SLE include cognitive dysfunction, mood disorder (including depression), cerebrovascular disease, seizures, polyneuropathy, anxiety disorder, cerebritis, and psychosis. CNS lupus can rarely present with intracranial hypertension syndrome, characterized by an elevated intracranial pressure, papilledema, and headache with occasional abducens nerve paresis, absence of a space-occupying lesion or ventricular enlargement, and normal cerebrospinal fluid chemical and hematological constituents. More rare manifestations are acute confusional state, Guillain-Barre syndrome, aseptic meningitis, autonomic disorder, demyelinating syndrome, mononeuropathy (which might manifest as mononeuritis multiplex), movement disorder (more specifically, chorea), myasthenia gravis, myelopathy, cranial neuropathy and plexopathy. Neural symptoms contribute to a significant percentage of morbidity and mortality in patients with lupus. As a result, the neural side of lupus is being studied in hopes of reducing morbidity and mortality rates. The neural manifestation of lupus is known as neuropsychiatric systemic lupus erythematosus (NPSLE). One aspect of this disease is severe damage to the epithelial cells of the blood-brain barrier.

SLE causes an increased rate of fetal death in utero and spontaneous abortion (miscarriage). The overall live-birth rate in SLE patients has been estimated to be 72%. Pregnancy outcome appears to be worse in SLE patients whose disease flares up during pregnancy. Neonatal lupus is the occurrence of SLE symptoms in an infant born from a mother with SLE, most commonly presenting with a rash resembling discoid lupus erythematosus, and sometimes with systemic abnormalities such as heart block or hepatosplenomegaly. Neonatal lupus is usually benign and self-limited.

Fatigue in SLE is probably multifactorial and has been related to not only disease activity or complications such as anemia or hypothyroidism, but also to pain, depression, poor sleep quality, poor physical fitness and lack of social support.

Different clinical measurements have been used to determine whether a SLE patients is having a clinic flare. One of the most common measurements is the SELENA-SLEDAI Flare Index (SFI). This scale uses a point system to calculate when the accumulated significance of recent changes in various indicators translates into a mild/moderate (SELENA-SLEDAI Index of 3-11 point change) or a severe (12 of more point change) flare, encompassing a change in disease activity score in conjunction with changes in organ system manifestations, treatments, and/or the physician's global assessment (PGA). Although helpful in defining clinical flares in therapeutic and observational SLE clinical trials, this information only defines a flare state and does not help predict or identify patients who are likely have an impending flare (an important clinical problem) and who would benefit from early intervention to prevent permanent organ damage. In addition, no consensus, objective molecular test or tests are consistently associated individually with increased disease activity, nor with imminent SLE disease flare. Having such a molecular test would be greatly beneficial to SLE clinical care to help guide therapy, prevent damage, and minimize therapeutic toxicity.

### Disease Classification

Antinuclear antibody (ANA) testing, anti-dsDNA, and anti-extractable nuclear antigen (anti-ENA) responses form the mainstay of SLE serologic testing. Several techniques are used to detect ANAs. Clinically the most widely used method is indirect immunofluorescence. The pattern of fluorescence suggests the type of antibody present in the patient's serum. Direct immunofluorescence can detect deposits of immunoglobulins and complement proteins in the patient's skin. When skin not exposed to the sun is tested, a positive direct IF (the so-called Lupus band test) is an evidence of systemic lupus erythematosus.

ANA screening yields positive results in many connective tissue disorders and other autoimmune diseases, and may occur in healthy individuals. Subtypes of antinuclear antibodies include anti-Smith and anti-double stranded DNA (dsDNA) antibodies (which are linked to SLE) and anti-histone antibodies (which are linked to drug-induced lupus). Anti-dsDNA antibodies are relatively specific for SLE; they are present in up to 50% of cases depending on ethnicity, whereas they appear in less than 2% of people without SLE. The anti-dsDNA antibody titers also tend to reflect disease activity, although not in all cases. Other ANA that may occur in SLE sufferers are anti-U1 RNP (which also appears in systemic sclerosis), anti-Ro (or anti-SSA) and anti-La (or anti-SSB; both of which are more common in primary Sjogren's syndrome). Anti-Ro and anti-La, when present in the maternal circulation, confer an increased risk for heart conduction block in neonatal lupus. Other tests routinely performed in suspected SLE are complement system levels (low levels suggest consumption by the immune system), electrolytes and renal function (disturbed if the kidneys are involved), liver enzymes, urine tests (proteinuria, hematuria, pyuria, and casts), complete blood count, and imaging studies.

### Biomarkers for Impending SLE Flares

### Flare Markers

In some embodiments, the biomarker comprises an innate cytokine. Innate cytokines are mediators secreted in response to immune system danger signals, such as toll like receptors (TLR). Innate cytokines which activate and are secreted by multiple immune cell types include Type I interferons (IFN-α and IFN-β), TNF-α, and members of the IL-1 family (IL-1α and IL-1β). Other innate cytokines, secreted by antigen presenting cells (APC), including dendritic cells, macrophages, and B-cells, as they process and present protein fragments (antigens, either from infectious agents or self-proteins that underly autoimmune disease) to CD4 T-helper (Th) cells, drive the development of antigen specific inflammatory pathways during the adaptive response, described below.

Th1-type cytokines Th1-type cytokines are associated with pro-inflammatory responses responsible for killing intracellular parasites and for perpetuating autoimmune responses. Excessive pro-inflammatory responses can lead to uncontrolled tissue damage, particularly in systemic lupus erythematosus (SLE).

CD4 Th cells differentiate to Th-1 type cells upon engagement of APC, co-stimulatory molecules, and APC-secreted cytokines, the hallmark of which is IL-12. IL-12 is composed of a bundle of four alpha helices. It is a heterodimeric cytokine encoded by two separate genes, IL-12A (p35) and IL-12B (p40). The active heterodimer, and a homodimer of p40, are formed following protein synthesis. IL-12 binds to the heterodimeric receptor formed by IL-12R-β1 and IL-12R-β2. IL-12R-β2 is considered to play a key role in IL-12 function, as it is found on activated T cells and is stimulated by cytokines that promote Th1 cell development and inhibited by those that promote Th2 cell development. Upon binding, IL-12R-β2 becomes tyrosine phosphorylated and provides binding sites for kinases, Tyk2 and Jak2. These are important in activating critical transcription factor proteins such as STAT4 that are implicated in IL-12 signaling in T cells and NK cells. IL-12 mediated signaling results in the production of interferon-gamma (IFN-γ) and tumor necrosis factor-alpha (TNF-α) from T and natural killer (NK) cells, and reduces IL-4 mediated suppression of IFN-γ.

IFNγ, or type II interferon, consists of a core of six α-helices and an extended unfolded sequence in the C-terminal region. IFNγ is critical for innate (NK cell) and adaptive (T cell) immunity against viral (CD8 responses) and intracellular bacterial (CD4 Th1 responses) infections and for tumor control. During the effector phase of the immune response, IFNγ activates macrophages. Aberrant IFNγ expression is associated with a number of auto-inflammatory and autoimmune diseases, including increased disease activity in SLE.

Although IFNγ is considered to be a characteristic Th1 cytokine, in humans, interleukin-2 (IL-2) can also influence Th1 differentiation, as well as its role as the predominant cytokine secreted during a primary response by naive Th cells. IL-2 is necessary for the growth, proliferation, and differentiation of T cells to become ‘effector’ T cells. IL-2 is normally produced by T cells during an immune response. Antigen binding to the T cell receptor (TCR) stimulates the secretion of IL-2, and the expression of IL-2 receptors IL-2R. The IL-2/IL-2R interaction then stimulates the growth, differentiation and survival of antigen-specific CD4⁺ T cells and CD8⁺ T cells. As such, IL-2 is necessary for the development of T cell immunologic memory, which depends upon the expansion of the number and function of antigen-selected T cell clones. IL-2, along with IL-7 and IL-15 (all members of the common cytokine receptor gamma-chain family), maintain lymphoid homeostasis to ensure a consistent number of lymphocytes during cellular turnover.

Th2-type cytokines. Th2-type cytokines include IL-4, IL-5, IL-13, as well as IL-6 (in humans), and are associated with the promotion of B-lymphocyte activation, antibody production, and isotype switching to IgE and eosinophilic responses in atopy. In excess, Th2 responses counteract the Th1 mediated microbicidal action. Th2-type cytokines may also contribute to SLE pathogenesis and increased disease activity.

IL-4 is a 15-kD polypeptide with multiple effects on many cell types. Its receptor is a heterodimer composed of an a subunit, with IL-4 binding affinity, and the common yγ subunit which is also part of other cytokine receptors. In T cells, binding of IL-4 to its receptor induces proliferation and differentiation into Th2 cells. IL-4 also contributes to the Th2-mediated activation of B-lymphocytes, antibody production, and, along with IL-5 and IL-13, isotype switching away from Th1-type isotypes (including IgG1 and IgG2) toward Th2-type isotypes (including IgG4, and IgE that contributes to atopy). In addition to its contributions to Th2 biology, IL-4 plays a significant role in immune cell hematopoiesis, with multiple effects on hematopoietic progenitors, including proliferation and differentiation of committed as well as primitive hematopoietic progenitors. It acts synergistically with granulocyte-colony stimulating factor (G-CSF) to support neutrophil colony formation, and, along with IL-1 and IL-6, induces the colony formation of human bone marrow B lineage cells.

IL-5 is an interleukin produced by multiple cell types, including Th2 cells, mast cells, and eosinophils. IL-5 expression is regulated by several transcription factors including GATA3. IL-5 is a 115-amino acid (in human; 133 in the mouse)-long TH2 cytokine that is part of the hematopoietic family. Unlike other members of this cytokine family (namely IL-3 and GM-CSF), this glycoprotein in its active form is a homodimer. Through binding to the IL-5 receptor, IL-5 stimulates B cell growth and increases immunoglobulin secretion. IL-5 has long been associated with the cause of several allergic diseases including allergic rhinitis and asthma, where mast cells play a significant role, and a large increase in the number of circulating, airway tissue, and induced sputum eosinophils have been observed.

Given the high concordance of eosinophils and, in particular, allergic asthma pathology, it has been widely speculated that eosinophils have an important role in the pathology of this disease. IL-13 is secreted by many cell types, but especially Th2 cells as a mediator of allergic inflammation and autoimmune disease, including type 1 diabetes mellitus, rheumatoid arthritis (RA) and SLE. IL-13 induces its effects through a multi-subunit receptor that includes the alpha chain of the IL-4 receptor (IL-4Ra) and at least one of two known IL-13-specific binding chains. Most of the biological effects of IL-13, like those of IL-4, are linked to a single transcription factor, signal transducer and activator of transcription 6 (STAT6).

Like IL-4, IL-13 is known to induce changes in hematopoietic cells, but to a lesser degree. IL-13 can induce immunoglobulin E (IgE) secretion from activated human B cells. IL-13 induces many features of allergic lung disease, including airway hyperresponsiveness, goblet cell metaplasia and mucus hypersecretion, which all contribute to airway obstruction. IL-4 contributes to these physiologic changes, but to a lesser extent than IL-13. IL-13 also induces secretion of chemokines that are required for recruitment of allergic effector cells to the lung.

IL-13 may antagonize Th 1 responses that are required to resolve intracellular infections and induces physiological changes in parasitized organs that are required to expel the offending organisms or their products. For example, expulsion from the gut of a variety of mouse helminths requires IL-13 secreted by Th2 cells. IL-13 induces several changes in the gut that create an environment hostile to the parasite, including enhanced contractions and glycoprotein hyper-secretion from gut epithelial cells, that ultimately lead to detachment of the organism from the gut wall and their removal.

Interleukin 6 (IL-6) is secreted by multiple cell types and participates in multiple innate and adaptive immune response pathways. IL-6 mediates its biological functions through a signal-transducing component of the IL-6 receptor (IL-6R), gp130, that leads to tyrosine kinase phosphorylation and downstream signaling events, including the STAT1/3 and the SHP2/ERK cascades. IL-6 is a key mediator of fever and stimulates an acute phase response during infection and after trauma. It is capable of crossing the blood brain barrier and initiating synthesis of PGE2 in the hypothalamus, thereby changing the body's temperature setpoint. In muscle and fatty tissue, IL-6 stimulates energy mobilization which leads to increased body temperature.

IL-6 can be secreted by multiple immune cells in response to specific microbial molecules, referred to as pathogen associated molecular patterns (PAMPs). These PAMPs bind to highly important group of detection molecules of the innate immune system, called pattern recognition receptors (PRRs), including Toll-like receptors (TLRs). These are present on the cell surface and intracellular compartments and induce intracellular signaling cascades that give rise to inflammatory cytokine production. As a Th2-type cytokine in humans, IL-6, along with IL-4, IL-5, and IL-13, can influence IgE production and eosinophil airway infiltration in asthma. IL-6 also contributes to Th2-type adaptive immunity against parasitic infections, with particular importance in mast-cell activation that coincides with parasite expulsion.

IL-6 is also a Th17-type cytokine, driving IL-17 production by T-lymphocytes in conjunction with TGF-β. IL-6 sensitizes Th17 cells to IL-23 (produced by APC) and IL-21 (produced by T-lymphocytes to perpetuate the Th17 response. Th17-type responses are described below. Th17-type cytokines. Th17 cells are a subset of T helper cells are considered developmentally distinct from Th1 and Th2 cells and excessive amounts of the cell are thought to play a key role in autoimmune disease, such as multiple sclerosis (which was previously thought to be caused solely by Th1 cells), psoriasis, autoimmune uveitis, Crohn's disease, type 2 diabetes mellitus, rheumatoid arthritis, and SLE. Th17 are thought to play a role in inflammation and tissue injury in these conditions. In addition to autoimmune pathogenesis, Th17 cells serve a significant function in anti-microbial immunity at epithelial/mucosal barriers. They produce cytokines (such as IL-21 and IL-22) that stimulate epithelial cells to produce anti-microbial proteins for clearance of microbes such as Candida and Staphylococcus species. A lack of Th17 cells may leave the host susceptible to opportunistic infections. In addition to its role in autoimmune disease and infection, the Th17 pathway has also been implicated in asthma, including the recruitment of neutrophils to the site of airway inflammation.

Interleukin 17A (IL-17A), is the founding member of a group of cytokines called the IL-17 family. Known as CTLA8 in rodents, IL-17 shows high homology to viral IL-17 encoded by an open reading frame of the T-lymphotropic rhadinovirus Herpesvirus saimiri. IL-17A is a 155-amino acid protein that is a disulfide-linked, homodimeric, secreted glycoprotein with a molecular mass of 35 kDa. Each subunit of the homodimer is approximately 15-20 kDa. The structure of IL-17A consists of a signal peptide of 23 amino acids followed by a 123-residue chain region characteristic of the IL-17 family. An N-linked glycosylation site on the protein was first identified after purification of the protein revealed two bands, one at 15 KDa and another at 20 KDa. Comparison of different members of the IL-17 family revealed four conserved cysteines that form two disulfide bonds. IL-17A is unique in that it bears no resemblance to other known interleukins. Furthermore, IL-17A bears no resemblance to any other known proteins or structural domains.

The crystal structure of IL-17F, which is 50% homologous to IL-17A, revealed that IL-17F is structurally similar to the cysteine knot family of proteins that includes the neurotrophins. The cysteine knot fold is characterized by two sets of paired β-strands stabilized by three disulfide interactions. However, in contrast to the other cysteine knot proteins, IL-17F lacks the third disulfide bond. Instead, a serine replaces the cysteine at this position. This unique feature is conserved in the other IL-17 family members. IL-17F also dimerizes in a fashion similar to nerve growth factor (NGF) and other neurotrophins.

IL-17A acts as a potent mediator in delayed-type reactions by increasing chemokine production in various tissues to recruit monocytes and neutrophils to the site of inflammation, similar to IFNγ. IL-17A is produced by T-helper cells and is induced by APC production of IL-6 (and TGF-β) and IL-23, resulting in destructive tissue damage in delayed-type reactions. IL-17 as a family functions as a proinflammatory cytokine that responds to the invasion of the immune system by extracellular pathogens and induces destruction of the pathogen's cellular matrix. IL-17 acts synergistically with TNF-α and IL-1. To elicit its functions, IL-17 binds to a type I cell surface receptor called IL-17R of which there are at least three variants IL17RA, IL17RB, and IL17RC.

IL-23 is produced by APC, including dendritic cells, macrophages, and B cells. The IL-23A gene encodes the p19 subunit of the heterodimeric cytokine. IL-23 is composed of this protein and the p40 subunit of IL-12. The receptor of IL-23 is formed by the beta 1 subunit of IL-12 (IL-12β1) and an IL-23 specific subunit, IL-23R. While IL-12 stimulates IFNγ production via STAT4, IL-23 primarily stimulates IL-17 production via STAT3 in conjunction with IL-6 and TGF-β.

IL-21 is expressed in activated human CD4+T cells, most notably Th17 cells and T follicular helper (Tfh) cells. IL-21 is also expressed in NK T cells. IL-21 has potent regulatory effects on cells of the immune system, including natural killer (NK) cells and cytotoxic T cells that can destroy virally infected or cancerous cells. This cytokine induces cell division/proliferation in its target cells.

The IL-21 receptor (IL-21R) is expressed on the surface of T, B and NK cells. Belonging to the common cytokine receptor gamma-chain family, IL-21R requires dimerization with the common gamma chain (γc) in order to bind IL-21. When bound to IL-21, the IL-21 receptor acts through the Jak/STAT pathway, utilizing Jak1 and Jak3 and a STAT3 homodimer to activate its target genes.

IL-21 may be a critical factor in the control of persistent viral infections. IL-21 (or IL-21R) knock-out mice infected with chronic LCMV (lymphocytic choriomeningitis virus) were not able to overcome chronic infection compared to normal mice. Besides, these mice with impaired IL-21 signaling had more dramatic exhaustion of LCMV-specific CD8+ T cells, suggesting that IL-21 produced by CD4⁺ T cells is required for sustained CD8+ T cell effector activity and then, for maintaining immunity to resolve persistent viral infection. Thus, IL-21 may contribute to the mechanism by which CD4⁺ T helper cells orchestrate the immune system response to viral infections.

In addition to promoting Th17 responses that contribute to chronic inflammation and tissue damage in autoimmune disease, IL-21 induces Tfh cell formation within the germinal center and signals directly to germinal center B cells to sustain germinal center formation and its response. IL-21 also induces the differentiation of human naive and memory B cells into antibody secreting plasma cells, thought to play a key role in autoantibody production in SLE.

Additionally or alternatively, in some embodiments, the biomarker comprises a chemokines and adhesion molecules. Chemokines and adhesion molecules (in this case, ICAM-1 and E-selectin) serve to coordinate cellular traffic within the immune response. Chemokines are divided into CXC (R)eceptor/CXC (L)igand and CCR/CCL subgroups.

GROα, also known as Chemokine (C-X-C motif) ligand 1 (CXCL1) is belongs to the CXC chemokine family that was previously called GRO1 oncogene, KC, Neutrophil-activating protein 3 (NAP-3) and melanoma growth stimulating activity, alpha (MSGA-α). In humans, this protein is encoded by the CXCL1 gene on chromosome 4. CXCL1 is expressed by macrophages, neutrophils and epithelial cells, and has neutrophil chemoattractant activity. GROα is involved in the processes of angiogenesis, inflammation, wound healing, and tumorigenesis. This chemokine elicits its effects by signaling through the chemokine receptor CXCR2.

Interleukin 8 (IL-8)/CXCL8 is a chemokine produced by macrophages and other cell types such as epithelial cells, airway smooth muscle cells and endothelial cells. In humans, the interleukin-8 protein is encoded by the IL8 gene. IL-8 is a member of the CXC chemokine family. The genes encoding this and the other ten members of the CXC chemokine family form a cluster in a region mapped to chromosome 4q.

There are many receptors of the surface membrane capable to bind IL-8; the most frequently studied types are the G protein-coupled serpentine receptors CXCR1, and CXCR2, expressed by neutrophils and monocytes. Expression and affinity to IL-8 is different in the two receptors (CXCR1>CXCR2). IL-8 is secreted and is an important mediator of the immune reaction in the innate immunity in response to TLR engagement. During the adaptive immune response, IL-8 is produced during the effector phase of Th1 and Th17 pathways, resulting in neutrophil and macrophage recruitment to sites of inflammation, including inflammation during infection and autoimmune disease. While neutrophil granulocytes are the primary target cells of IL-8, there are a relative wide range of cells (endothelial cells, macrophages, mast cells, and keratinocytes) also responding to this chemokine.

Monokine induced by y-interferon (MIG)/CXCL9 is a T-cell chemoattractant induced by IFN-γ. It is closely related to two other CXC chemokines, IP-10/CXCL10 and I-TAC/CXCL11, whose genes are located near the CXCL9 gene on human chromosome 4. MIG, IP-10, and I-TAC elicit their chemotactic functions by interacting with the chemokine receptor CXCR3.

Interferon gamma-induced protein 10 (IP-10), also known as CXCL10, or small-inducible cytokine B10, is an 8.7 kDa protein that in humans is encoded by the CXCL10 gene located on human chromosome 4 in a cluster among several other CXC chemokines. IP10 is secreted by several cell types in response to IFN-y. These cell types include monocytes, endothelial cells and fibroblasts. IP-10 has been attributed to several roles, such as chemoattraction for monocytes/macrophages, T cells, NK cells, and dendritic cells, promotion of T cell adhesion to endothelial cells, antitumor activity, and inhibition of bone marrow colony formation and angiogenesis. This chemokine elicits its effects by binding to the cell surface chemokine receptor CXCR3, which can be found on both Th1 and Th2 cells.

Monocyte chemotactic protein-1 (MCP-1)/CCL2 recruits monocytes, memory T cells, and dendritic cells to sites of inflammation. MCP-1 is a monomeric polypeptide, with a molecular weight of approximately 13 kDa that is primarily secreted by monocytes, macrophages and dendritic cells. Platelet derived growth factor is a major inducer of MCP-1 gene. The MCP-1 protein is activated post-cleavage by metalloproteinase MMP-12. CCR2 and CCR4 are two cell surface receptors that bind MCP-1. During the adaptive immune response, CCR2 is upregulated on Th17 and T-regulatory cells, while CCR4 is upregulated on Th2 cells. MCP-1 is implicated in pathogeneses of several diseases characterized by monocytic infiltrates, such as psoriasis, rheumatoid arthritis and atherosclerosis. It is also implicated in the pathogenesis of SLE and a polymorphism of MCP-1 is linked to SLE in Caucasians. Administration of anti-MCP-1 antibodies in a model of glomerulonephritis reduces infiltration of macrophages and T cells, reduces crescent formation, as well as scarring and renal impairment.

Monocyte-specific chemokine 3 (MCP-3)/CCL7) specifically attracts monocytes and regulates macrophage function. It is produced by multiple cell types, including monocytes, macrophages, and dendritic cells. The CCL7 gene is located on chromosome 17 in humans, in a large cluster containing other CC chemokines. MCP-3 is most closely related to MCP-1, binding to CCR2.

Macrophage inflammatory protein-1α (MIP-1α)/CCL3 is encoded by the CCL3 gene in humans. MIP-1α is involved in the acute inflammatory state in the recruitment and activation of polymorphonuclear leukocytes. MIP-1α interacts with MIP-1β/CCL4, encoded by the CCL4 gene, with specificity for CCR5 receptors. It is a chemoattractant for natural killer cells, monocytes and a variety of other immune cells.

RANTES (Regulated on Activation, Normal T cell Expressed and Secreted)/CCL5 is encoded by the CCLS gene on chromosome 17 in humans. RANTES is an 8 kDa protein chemotactic for T cells, eosinophils, and basophils, playing an active role in recruiting leukocytes to sites of inflammation. With the help of particular cytokines that are released by T cells (e.g. IL-2 and IFN-γ), RANTES induces the proliferation and activation of natural-killer (NK) cells. RANTES was first identified in a search for genes expressed “late” (3-5 days) after T cell activation and has been shown to interact with CCR3, CCR5 and CCR1. RANTES also activates the G-protein coupled receptor GPR75.

Eotaxin-1/CCL11 is a member of a CC chemokine subfamily of monocyte chemotactic proteins. In humans, there are three family members, CCL11 (eotaxin-1), CCL24 (eotaxin-2) and CCL26 (eotaxin-3). Eotaxin-1, also known as eosinophil chemotactic protein, is encoded by the CCL11 gene located on chromosome 17. Eotaxin-1 selectively recruits eosinophils and is implicated in allergic responses. The effects of Eotaxin-1 are mediated by its binding to G-protein-linked receptors CCR2, CCR3 and CCR5.

Soluble cell adhesion molecules (sCAMs) are a class of cell surface binding proteins that may represent important biomarkers for inflammatory processes involving activation or damage to cells such as platelets and the endothelium. They include soluble forms of the cell adhesion molecules ICAM-1, VCAM-1, E-selectin, L-selectin, and P-selectin (distinguished as sICAM-1, sVCAM-1, sE-selectin, sL-selectin, and sP-selectin). The cellular expression of CAMs is difficult to assess clinically, but these soluble forms are present in the circulation and may serve as markers for CAMs.

ICAM-1 (Intercellular Adhesion Molecule 1) also known as CD54, is encoded by the ICAM1 gene in humans. This gene encodes a cell surface glycoprotein which is typically expressed on endothelial cells and cells of the immune system. The protein encoded by this gene is a type of intercellular adhesion molecule continuously present in low concentrations in the membranes of leukocytes and endothelial cells. ICAM-1 can be induced by IL-1 and TNF-α, and is expressed by the vascular endothelium, macrophages, and lymphocytes.

The presence of heavy glycosylation and other structural characteristics of ICAM-1 lend the protein binding sites for numerous ligands. ICAM-1 possesses binding sites for a number of immune-associated ligands. Notably, ICAM-1 binds to macrophage adhesion ligand-1 (Mac-1; ITGB2/ITGAM), leukocyte function associated antigen-1 (LFA-1), and fibrinogen. These three proteins are generally expressed on endothelial cells and leukocytes, and they bind to ICAM-1 to facilitate transmigration of leukocytes across vascular endothelia in processes such as extravasation and the inflammatory response. As a result of these binding characteristics, ICAM-1 has classically been assigned the function of intercellular adhesion.

ICAM-1 is a member of the immunoglobulin superfamily, the superfamily of proteins, including B-cell receptors (membrane-bound antibodies) and T-cell receptors. In addition to its roles as an adhesion molecule, ICAM-1 has been shown to be a co-stimulatory molecule for the TCR on T-lymphocytes. The signal-transducing functions of ICAM-1 are associated primarily with proinflammatory pathways. In particular, ICAM-1 signaling leads to recruitment of inflammatory immune cells such as macrophages and granulocytes.

E-selectin, also known as CD62 antigen-like family member E (CD62E), endothelial-leukocyte adhesion molecule 1 (ELAM-1), or leukocyte-endothelial cell adhesion molecule 2 (LECAM2), is a cell adhesion molecule expressed on cytokine-activated endothelial cells. Playing an important role in inflammation, E-selectin is encoded by the SELE gene in humans. Its C-type lectin domain, EGF-like, SCR repeats, and transmembrane domains are each encoded by separate exons, whereas the E-selectin cytosolic domain derives from two exons. The E-selectin locus flanks the L-selectin locus on chromosome 1.

Different from P-selectin, which is stored in vesicles called Weibel-Palade bodies, E-selectin is not stored in the cell and has to be transcribed, translated, and transported to the cell surface. The production of E-selectin is stimulated by the expression of P-selectin which is stimulated by TNF-α, IL-1 and through engagement of TLR4 by LPS. It takes about two hours, after cytokine recognition, for E-selectin to be expressed on the endothelial cell's surface. Maximal expression of E-selectin occurs around 6-12 hours after cytokine stimulation, and levels returns to baseline within 24 hours.

E-selectin recognizes and binds to sialylated carbohydrates present on the surface proteins of leukocytes. E-selectin ligands are expressed by neutrophils, monocytes, eosinophils, memory-effector T-like lymphocytes, and natural killer cells. Each of these cell types is found in acute and chronic inflammatory sites in association with expression of E-selectin, thus implicating E-selectin in the recruitment of these cells to such inflammatory sites. These carbohydrates include members of the Lewis X and Lewis A families found on monocytes, granulocytes, and T-lymphocytes.

Additionally or alternatively, in some embodiments, the biomarker comprises a TNF Receptor superfamily member. The tumor necrosis factor receptor (TNFR) superfamily of receptors and their respective ligands activate signaling pathways for cell survival, death, and differentiation. Members of the TNFR superfamily act through ligand-mediated trimerization and require adaptor molecules (e.g. TRAFs) to activate downstream mediators of cellular activation, including NF-κB and MAPK pathways, immune and inflammatory responses, and in some cases, apoptosis.

In some embodiments, the TNF Receptor superfamily member is TNF-α. Tumor necrosis factor (TNF, cachexin, or cachectin, and formerly known as tumor necrosis factor alpha or TNF-α) is a cytokine involved in systemic inflammation and is a member of a group of cytokines that stimulate the acute phase reaction. It is produced by a number of immune cells, including macrophages, dendritic cells, and both T- and B -lymphocytes. Dysregulation of TNF-α production has been implicated in a variety of human diseases including Alzheimer's disease, cancer, major depression and autoimmune disease, including inflammatory bowel disease (IBD) and rheumatoid arthritis (RA).

TNF-α is produced as a 212-amino acid-long type II transmembrane protein arranged in stable homotrimers. From this membrane-integrated form the soluble homotrimeric cytokine (sTNF) is released via proteolytic cleavage by the metalloprotease TNF-α converting enzyme (TACE, also called ADAM17). The soluble 51 kDa trimeric sTNF may dissociate to the 17-kD monomeric form. Both the secreted and the membrane bound forms are biologically active. Tumor necrosis factor receptor 1 (TNFRI; TNFRSF1 a; CD120a), is a trimeric cytokine receptor that is expressed in most tissues and binds both membranous and soluble TNF-α. The receptor cooperates with adaptor molecules (such as TRADD, TRAF, RIP), which is important in determining the outcome of the response (e.g., apoptosis, inflammation). Tumor necrosis factor II (TNFRII; TNFRSF1b; CD120b) has limited expression, primarily on immune cells (although during chronic inflammation, endothelial cells, including those of the lung and kidney, are induced to express TNFRII) and binds the membrane-bound form of the TNF-α homotrimer with greater affinity and avidity than soluble TNF-α. Unlike TNFRI, TNFRII does not contain a death domain (DD) and does not cause apoptosis, but rather contributes to the inflammatory response and acts as a co-stimulatory molecule in receptor-mediated B- and T-lymphocyte activation.

Fas, also known as apoptosis antigen 1 (APO-1 or APT), cluster of differentiation 95 (CD95) or tumor necrosis factor receptor superfamily member 6 (TNFRSF6) is a protein that in humans is encoded by the TNFRSF6 gene located on chromosome 10 in humans and 19 in mice. Fas is a death receptor on the surface of cells that leads to programmed cell death (apoptosis). Like other TNFR superfamily members, Fas is produced in membrane-bound form, but can be produced in soluble form, either via proteolytic cleavage or alternative splicing. The mature Fas protein has 319 amino acids, has a predicted molecular weight of 48 kD and is divided into 3 domains: an extracellular domain, a transmembrane domain, and a cytoplasmic domain. Fas forms the death-inducing signaling complex (DISC) upon ligand binding. Membrane-anchored Fas ligand on the surface of an adjacent cell causes oligomerization of Fas. Upon ensuing death domain (DD) aggregation, the receptor complex is internalized via the cellular endosomal machinery. This allows the adaptor molecule FADD to bind the death domain of Fas through its own death domain.

FADD also contains a death effector domain (DED) near its amino terminus, which facilitates binding to the DED of FADD-like interleukin-1 beta-converting enzyme (FLICE), more commonly referred to as caspase-8. FLICE can then self-activate through proteolytic cleavage into p10 and p18 subunits, two each of which form the active heterotetramer enzyme. Active caspase-8 is then released from the DISC into the cytosol, where it cleaves other effector caspases, eventually leading to DNA degradation, membrane blebbing, and other hallmarks of apoptosis.

In most cell types, caspase-8 catalyzes the cleavage of the pro-apoptotic BH3-only protein Bid into its truncated form, tBid. BH-3 only members of the Bc1-2 family exclusively engage anti-apoptotic members of the family (Bc1-2, Bc1-xL), allowing Bak and Bax to translocate to the outer mitochondrial membrane, thus permeabilizing it and facilitating release of pro-apoptotic proteins such as cytochrome c and Smac/DIABLO, an antagonist of inhibitors of apoptosis proteins (IAPs).

Fas ligand (FasL; CD95L; TNFSF6) is a type-II transmembrane protein that belongs to the tumor necrosis factor (TNF) family. Its binding with its receptor induces apoptosis. FasL/Fas interactions play an important role in the regulation of the immune system and the progression of cancer. Soluble Fas ligand is generated by cleaving membrane-bound FasL at a conserved cleavage site by the external matrix metalloproteinase MMP-7.

Apoptosis triggered by Fas-Fas ligand binding plays a fundamental role in the regulation of the immune system. Its functions include T-cell homeostasis (the activation of T-cells leads to their expression of the Fas ligand.; T cells are initially resistant to Fas-mediated apoptosis during clonal expansion, but become progressively more sensitive the longer they are activated, ultimately resulting in activation-induced cell death (AICD)), cytotoxic T-cell activity (Fas-induced apoptosis and the perforin pathway are the two main mechanisms by which cytotoxic T lymphocytes induce cell death in cells expressing foreign antigens), immune privilege (cells in immune privileged areas such as the cornea or testes express Fas ligand and induce the apoptosis of infiltrating lymphocytes), maternal tolerance (Fas ligand may be instrumental in the prevention of leukocyte trafficking between the mother and the fetus, although no pregnancy defects have yet been attributed to a faulty Fas-Fas ligand system) and tumor counterattack (tumors may over-express Fas ligand and induce the apoptosis of infiltrating lymphocytes, allowing the tumor to escape the effects of an immune response).

CD154, also called CD40 ligand (CD40L), is a member of the TNF superfamily protein that is expressed primarily on activated T cells. CD40L binds to CD40 (TNFRSF4), which is constitutively expressed by antigen-presenting cells (APC), including dendritic cells, macrophages, and B cells. CD40L engagement of CD40 induces maturation and activation of dendritic cells and macrophages in association with T cell receptor stimulation by MHC molecules on the APC. CD40L regulates B cell activation, proliferation, antibody production, and isotype switching by engaging CD40 on the B cell surface. A defect in this gene results in an inability to undergo immunoglobulin class switch and is associated with hyper IgM syndrome. While CD40L was originally described on T lymphocytes, its expression has since been found on a wide variety of cells, including platelets, endothelial cells, and aberrantly on B lymphocytes during periods of chronic inflammation.

B-cell activating factor (BAFF) also known as B Lymphocyte Stimulator (BLyS), TNF- and APOL-related leukocyte expressed ligand (TALL-1), and CD27 is encoded by the TNFSF13C gene in humans. BLyS is a 285-amino acid long peptide glycoprotein which undergoes glycosylation at residue 124. It is expressed as a membrane-bound type II transmembrane protein on various cell types including monocytes, dendritic cells and bone marrow stromal cells. The transmembrane form can be cleaved from the membrane, generating a soluble protein fragment. This cytokine is expressed in B cell lineage cells, and acts as a potent B cell activator. It has been also shown to play an important role in the proliferation and differentiation of B cells.

BLyS is a ligand for receptors TNFRSF13B/TACI, TNFRSF17/BCMA, and TNFRSF13C/BAFFR. These receptors are expressed mainly on mature B lymphocytes and their expression varies in dependence of B cell maturation (TACI is also found on a subset of T-cells and BCMA on plasma cells). BAFF-R is involved in the positive regulation during B cell development. TACI binds BLyS with the least affinity; its affinity is higher for a protein similar to BLyS, called a proliferation-inducing ligand (APRIL). BCMA displays an intermediate binding phenotype and will bind to either BLyS or APRIL to varying degrees. Signaling through BAFF-R and BCMA stimulates B lymphocytes to undergo proliferation and to counter apoptosis. All these ligands act as homotrimers (i.e. three of the same molecule) interacting with homotrimeric receptors, although BAFF has been known to be active as either a hetero- or homotrimer.

Excessive level of BLyS causes abnormally high antibody production, results in systemic lupus erythmatosis, rheumatoid arthritis, and many other autoimmune diseases. Belimumab (Benlysta) is a monoclonal antibody developed by Human Genome Sciences and GlaxoSmithKline, with significant discovery input by Cambridge Antibody Technology, which specifically recognizes and inhibits the biological activity of B-Lymphocyte stimulator (BLyS) and is in clinical trials for treatment of Systemic lupus erythematosus and other auto-immune diseases. Blisibimod, a fusion protein inhibitor of BLyS, is in development by Anthera Pharmaceuticals, also primarily for the treatment of systemic lupus erythematosus.

A proliferation-inducing ligand (APRIL), or tumor necrosis factor ligand superfamily member 13 (TNFSF13), is a protein that in humans is encoded by the TNFSF13 gene. APRIL has also been designated CD256 (cluster of differentiation 256). The protein encoded by this gene is a member of the tumor necrosis factor ligand (TNF) ligand family. This protein is a ligand for TNFRSF13B/TACI and TNFRSF17/BCMA receptors. This protein and its receptor are both found to be important for B cell development. In vivo experiments suggest an important role for APRIL in the long-term survival of plasma cells in the bone marrow. Mice deficient in APRIL demonstrate a reduced ability to support plasma cell survival. In vitro experiments suggested that this protein may be able to induce apoptosis through its interaction with other TNF receptor family proteins such as TNFRSF6/FAS and TNFRSF14/HVEM. Three alternatively spliced transcript variants of this gene encoding distinct isoforms have been reported.

Additionally or alternatively, in some embodiments, the biomarker comprises some other flare factors. For example, Osteopontin (OPN) is a matricellular protein with diverse cellular functions. Its ability to facilitate Th1-type cytokine responses and promote cell-mediated immunity suggests a potential role in chronic inflammation and autoimmunity. In the methods disclosed herein, OPN may be highly informative and was ranked first in random forest variable importance.

Leptin is a 16-kDa protein hormone that plays a key role in regulating energy intake and expenditure, including appetite and hunger, metabolism, and behavior. It is one of a number of adipokines, including adiponectin and resistin. The reported rise in leptin following acute infection and chronic inflammation, including autoimmune disease, suggests that leptin actively participates in the immune response. Leptin levels increase in response to a number of innate cytokines, including TNF-α and IL-6. Leptin is a member of the cytokine family that includes IL-6, IL-12, and G-CSF. Leptin functions by binding to the leptin receptor, which is expressed by polymorphonuclear neutrophils, circulating leukocytes (including monocytes), and NK cells. Leptin influences the rise in the chemokine MCP-1, allowing for recruitment of monocytes and macrophages to sites of inflammation.

Stem Cell Factor (also known as SCF, kit-ligand, KL, or steel factor) is a cytokine that binds to the c-Kit receptor (CD117). SCF can exist both as a transmembrane protein and a soluble protein. This cytokine plays an important role in hematopoiesis (formation of blood cells), spermatogenesis, and melanogenesis. The gene encoding stem cell factor (SCF) is found on the 51 locus in mice and on chromosome 12q22-12q24 in humans. The soluble and transmembrane forms of the protein are formed by alternative splicing of the same RNA transcript.

The soluble form of SCF contains a proteolytic cleavage site in exon 6. Cleavage at this site allows the extracellular portion of the protein to be released. The transmembrane form of SCF is formed by alternative splicing that excludes exon 6. Both forms of SCF bind to c-Kit and are biologically active. Soluble and transmembrane SCF is produced by fibroblasts and endothelial cells. Soluble SCF has a molecular weight of 18.5 kDa and forms a dimer. SCF plays an important role in the hematopoiesis, providing guidance cues that direct hematopoietic stem cells (HSCs) to their stem cell niche (the microenvironment in which a stem cell resides), and it plays an important role in HSC maintenance. SCF plays a role in the regulation of HSCs in the stem cell niche in the bone marrow. SCF has been shown to increase the survival of HSCs in vitro and contributes to the self-renewal and maintenance of HSCs in vivo. HSCs at all stages of development express the same levels of the receptor for SCF (c-Kit). The stromal cells that surround HSCs are a component of the stem cell niche, and they release a number of ligands, including SCF.

A small percentage of HSCs regularly leave the bone marrow to enter circulation and then return to their niche in the bone marrow. It is believed that concentration gradients of SCF, along with the chemokine SDF-1, allow HSCs to find their way back to the niche.

In addition to hematopoiesis, SCF is thought to contribute to inflammation via its binding to c-kit on dendritic cells. This engagement leads to increased secretion of IL-6 and the promoted development of Th2 and Th17-type immune responses. Th2 cytokines synergize with SCF in the activation of mast cells, and integral promoter of allergic inflammation. The induction of IL-17 allows for further upregulation of SCF by epithelial cells and the promotion of granulopoiesis. In the lung, the upregulation of IL-17 induces IL-8 and MIP-2 to recruit neutrophils to the lung. The chronic induction of IL-17 has been demonstrated to play a role in autoimmune diseases, including multiple sclerosis and rheumatoid arthritis.

Matrix metallopeptidase 9 (MMP-9) is a zinc-metalloproteinase involved in extracellular matrix degradation, for example, in physiological process such as angiogenesis, bone development, wound healing, embryo development.

Tissue inhibitor of metalloproteinases (TIMP1) is a glycoprotein inhibitor of metalloproteinases involved in extracellular matrix degradation.

Additionally or alternatively, in some embodiments, the biomarker comprises a marker exhibiting depressed expression with impending flare.

IL-10. Interleukin-10 (IL-10), also known as human cytokine synthesis inhibitory factor (CSIF), is an anti-inflammatory cytokine. The IL-10 protein is a homodimer; each of its subunits is 178-amino-acid long. IL-10 is classified as a class-2 cytokine, a set of cytokines including IL-19, IL-20, IL-22, IL-24 (Mda-7), and IL-26, interferons and interferon-like molecules. In humans, IL-10 is encoded by the IL10 gene, which is located on chromosome 1 and comprises 5 exons. IL-10 is primarily produced by monocytes and lymphocytes, namely Th2 cells, CD4⁺, CD25⁺, Foxp3⁺, regulatory T cells, and in a certain subset of activated T cells and B cells. IL-10 can be produced by monocytes upon PD-1 triggering in these cells. The expression of IL-10 is minimal in unstimulated tissues and requires receptor-mediated cellular activation for its expression. IL-10 expression is tightly regulated at the transcriptional and post-transcriptional level. Extensive IL-10 locus remodeling is observed in monocytes upon stimulation of TLR or Fc receptor pathways. IL-10 induction involves ERK1/2, p38 and NFκB signalling and transcriptional activation via promoter binding of the transcription factors NFκB and AP-1. IL-10 may autoregulate its expression via a negative feed-back loop involving autocrine stimulation of the IL-10 receptor and inhibition of the p38 signaling pathway. Additionally, IL-10 expression is extensively regulated at the post-transcriptional level, which may involve control of mRNA stability via AU-rich elements and by microRNAs such as let-7 or miR-106.

IL-10 is a cytokine with pleiotropic effects in immunoregulation and inflammation. It downregulates the expression of multiple Th-pathway cytokines, MHC class II antigens, and co-stimulatory molecules on macrophages. It also enhances B cell survival, proliferation, and antibody production. IL-10 can block NF-κB activity, and is involved in the regulation of the JAK-STAT signaling pathway.

TGF-β. Transforming growth factor beta (TGF-β) controls proliferation, cellular differentiation, and other functions in most cells. TGF-β is a secreted protein that exists in at least three isoforms called TGF-β1, TGF-β2 and TGF-β3. It was also the original name for TGF-β1, which was the founding member of this family. The TGF-β family is part of a superfamily of proteins known as the transforming growth factor beta superfamily, which includes inhibins, activin, anti-mullerian hormone, bone morphogenetic protein, decapentaplegic and Vg-1.

Most tissues have high expression of the gene encoding TGF-β. That contrasts with other anti-inflammatory cytokines such as IL-10, whose expression is minimal in unstimulated tissues and seems to require triggering by commensal or pathogenic flora.

The peptide structures of the three members of the TGF-β family are highly similar. They are all encoded as large protein precursors; TGF-β1 contains 390 amino acids and TGF-β2 and TGF-β3 each contain 412 amino acids. They each have an N-terminal signal peptide of 20-30 amino acids that they require for secretion from a cell, a pro-region (called latency associated peptide or LAP), and a 112-114 amino acid C-terminal region that becomes the mature TGF-β molecule following its release from the pro-region by proteolytic cleavage. The mature TGF-β protein dimerizes to produce a 25 kDa active molecule with many conserved structural motifs.

TGF-β plays a crucial role in the regulation of the cell cycle. TGF-β causes synthesis of p15 and p21 proteins, which block the cyclin:CDK complex responsible for Retinoblastoma protein (Rb) phosphorylation. Thus TGF-β blocks advance through the G1 phase of the cycleTGF-β is necessary for CD4⁺, CD25⁺, Foxp3⁺, T-regulatory cell differentiation and suppressive function. In the presence of IL-6, TGF-β contributes to the differentiation of pro-inflammatory Th17 cells.

SDF-1. Stromal cell-derived factor 1 (SDF-1), also known as C-X-C motif chemokine 12 (CXCL12), is encoded by the CXCL12 gene on chromosome 10 in humans. SDF-1 is produced in two forms, SDF- 1α/CXCL12a and SDF-1β/CXCL12b, by alternate splicing of the same gene. Chemokines are characterized by the presence of four conserved cysteines, which form two disulfide bonds. The CXCL12 proteins belong to the group of CXC chemokines, whose initial pair of cysteines are separated by one intervening amino acid.

CXCL12 is strongly chemotactic for lymphocytes. During embryogenesis it directs the migration of hematopoietic cells from fetal liver to bone marrow and the formation of large blood vessels. CXCL12 knockout mice are embryonic lethal.

The receptor for this chemokine is CXCR4, which was previously called LESTR or fusin. This CXCL12-CXCR4 interaction was initially thought to be exclusive (unlike for other chemokines and their receptors), but recently it was suggested that CXCL12 may also bind the CXCR7 receptor. The CXCR4 receptor is a G-Protein Coupled Receptor that is widely expressed, including on T-regulatory cells, allowing them to be recruited to promote lymphocyte homeostasis and immune tolerance. In addition to CXCL12, CXCR4 binds Granulocyte-Colony Stimulating Factor (G-CSF). G-CSF binds CXCR4 to prevent SDF-1 binding, which results in the inhibition of the pathway.

IL-1RA. The interleukin-1 receptor antagonist (IL-1RA) is a protein that in humans is encoded by the IL1RN gene. A member of the IL-1 cytokine family, IL-1RA, is an agent that binds non-productively to the cell surface interleukin-1 receptor (IL-1R), preventing IL-1 from binding and inducing downstream signaling events.

IL1Ra is secreted by various types of cells including immune cells, epithelial cells, and adipocytes, and is a natural inhibitor of the pro-inflammatory effect of IL-1α and IL1β. This gene and five other closely related cytokine genes form a gene cluster spanning approximately 400 kb on chromosome 2. Four alternatively spliced transcript variants encoding distinct isoforms have been reported.

An interleukin 1 receptor antagonist is used in the treatment of rheumatoid arthritis, an autoimmune disease in which IL-1 plays a key role. It is commercially produced as anakinra, which is a human recombinant form of IL-1RA Anakinra has shown both safety and efficacy in improving arthritis in an open trial on four SLE patients, with only short-lasting therapeutic effects in two patients.

In some embodiments, assessing biomarker expression may be assessed according to any suitable method. Generally, the principle applications are to (a) determine if a patient has SLE as opposed to a distinct autoimmune condition, (b) to determine the severity of the disease, (c) to determine the current intensity of the inflammatory state, (d) to predict or assess an impending disease flare, and (e) to predict or assess the efficacy of a therapy. In each of these assays, the expression of various biomarkers will be measured, and in some, the expression is measured multiple times to assess not only absolute values, but changes in these values overtime. Virtually any method of measuring gene expression may be utilized, and the following discussion is exemplary in nature and in no way limiting.

In some embodiments, biomarker expression is assessed via an immunologic assay. There are a variety of methods that can be used to assess protein expression. One such approach is to perform protein identification with the use of antibodies. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” also refers to any antibody-like molecule that has an antigen binding region, and includes antibody fragments such as Fab′, Fab, F(ab′)₂, single domain antibodies (DAB s), Fv, scFv (single chain Fv), and the like. The techniques for preparing and using various antibody-based constructs and fragments are well known in the art. Means for preparing and characterizing antibodies, both polyclonal and monoclonal, are also well known in the art (see, e.g., Antibodies: A Laboratory Manual, Cold Spring Harbor Laboratory, 1988; incorporated herein by reference). In particular, antibodies to calcyclin, calpactin I light chain, astrocytic phosphoprotein PEA-15 and tubulin-specific chaperone A are contemplated.

In some embodiments, immunodetection methods are provided. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle and Ben-Zeev O, 1999; Gulbis and Galand, 1993; De Jager et al., 1993; and Nakamura et al., 1987, each incorporated herein by reference.

In general, the immunobinding methods include obtaining a sample suspected of containing a relevant polypeptide, and contacting the sample with a first antibody under conditions effective to allow the formation of immunocomplexes. In terms of antigen detection, the biological sample analyzed may be any sample that is suspected of containing an antigen, such as, for example, a tissue section or specimen, a homogenized tissue extract, a cell, or even a biological fluid.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

As detailed above, immunoassays are in essence binding assays. Certain immunoassays are the various types of enzyme linked immunosorbent assays (ELISAs) and radioimmunoassays (RIA) known in the art. However, it will be readily appreciated that detection is not limited to such techniques, and Western blotting, dot blotting, FACS analyses, and the like may also be used.

In one exemplary ELISA, the antibodies are immobilized onto a selected surface exhibiting protein affinity, such as a well in a polystyrene microtiter plate. Then, a test composition suspected of containing the antigen, such as a clinical sample, is added to the wells. After binding and washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and then contacted with the anti-ORF message and anti-ORF translated product antibodies. After binding and washing to remove non-specifically bound immune complexes, the bound anti-ORF message and anti-ORF translated product antibodies are detected. Where the initial anti-ORF message and anti-ORF translated product antibodies are linked to a detectable label, the immune complexes may be detected directly. Again, the immune complexes may be detected using a second antibody that has binding affinity for the first anti-ORF message and anti-ORF translated product antibody, with the second antibody being linked to a detectable label.

Another ELISA in which the antigens are immobilized, involves the use of antibody competition in the detection. In this ELISA, labeled antibodies against an antigen are added to the wells, allowed to bind, and detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background. The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Another antibody-based approach to assessing biomarkers expression is Fluorescence-Activated Cell Sorting (FACS), a specialized type of flow cytometry. It provides a method for sorting a heterogeneous mixture of biological cells into two or more containers, one cell at a time, based upon the specific light scattering and fluorescent characteristics of each cell. It provides fast, objective and quantitative recording of fluorescent signals from individual cells as well as physical separation of cells of particular interest. A cell suspension is entrained in the center of a narrow, rapidly flowing stream of liquid. The flow is arranged so that there is a large separation between cells relative to their diameter. A vibrating mechanism causes the stream of cells to break into individual droplets. The system is adjusted so that there is a low probability of more than one cell per droplet. Just before the stream breaks into droplets, the flow passes through a fluorescence measuring station where the fluorescent character of interest of each cell is measured. An electrical charging ring is placed just at the point where the stream breaks into droplets. A charge is placed on the ring based on the immediately prior fluorescence intensity measurement, and the opposite charge is trapped on the droplet as it breaks from the stream. The charged droplets then fall through an electrostatic deflection system that diverts droplets into containers based upon their charge. In some systems, the charge is applied directly to the stream, and the droplet breaking off retains charge of the same sign as the stream. The stream is then returned to neutral after the droplet breaks off. One common way to use FAC is with a fluorescently labeled antibody that binds to a target on or in a cell, thereby identifying cells with a given target. This technique can be used quantitatively where the amount of fluorescent activity correlates to the amount of target, thereby permitting one to sort based on relative amounts of fluorescence, and hence relative amounts of the target.

Bead-based xMAP Technology may also be applied to immunologic detection in conjunction with the presently disclosed subject matter. This technology combines advanced fluidics, optics, and digital signal processing with proprietary microsphere technology to deliver multiplexed assay capabilities. Featuring a flexible, open-architecture design, xMAP technology can be configured to perform a wide variety of bioassays quickly, cost-effectively and accurately.

Fluorescently-coded microspheres are arranged in up to 500 distinct sets. Each bead set can be coated with a reagent specific to a particular bioassay (e.g., an antibody), allowing the capture and detection of specific analytes from a sample, such as the biomarkers of the present application. Inside the xMAP multiplex analyzer, a light source excites the internal dyes that identify each microsphere particle, and also any reporter dye captured during the assay. Many readings are made on each bead set, which further validates the results. Using this process, xMAP Technology allows multiplexing of up to 500 unique bioassays within a single sample, both rapidly and precisely. Unlike other flow cytometer microsphere-based assays which use a combination of different sizes and color intensities to identify an individual microsphere, xMAP technology uses 5.6 micron size microspheres internally dyed with red and infrared fluorophores via a proprietary dying process to create 500 unique dye mixtures which are used to identify each individual microsphere.

Some of the advantages of xMAP include multiplexing (reduces costs and labor), generation of more data with less sample, less labor and lower costs, faster, more reproducible results than solid, planar arrays, and focused, flexible multiplexing of 1 to 500 analytes to meet a wide variety of applications.

An automated microfluidic immunoassay platform may also be applied to immunologic detection in conjunction with the presently disclosed subject matter. This technology manipulates small volumes of fluid and flow to miniaturize quantitative measurement of multiple protein biomarkers (multiplexing) in parallel, thus minimizing cross-biomarker interactions that can lead to false negative (inhibitory) and false positive (enhanced) measurements. The consumption of reagent and sample volume is low due to miniaturized design, and therefore researchers are able to conserve precious samples. Microfluidics may provide for automation of processes including preparation, incubation, and detection all on one device (or lab-on-a-chip) so that complex lab procedures and inter-user variability can be minimized.

Capture antibodies for targeted analytes are coupled to nanoreactors in each assay channel within a standardized cartridge wherein automated exposure to samples, wash buffers, detection antibodies, and fluorescent labels occurs, for example, in triplicate. A fluorescent detection system may evaluate the amount of assay signal against a pre-determined, lot-specific standard curve to quantify the amount of signal (analyte/biomarker) within a given assayed sample.

Advantages of this automated technology include high reproducibility (for example, no pipetting errors), limited inter-user and inter-testing site variability (immunoassay platform is climate controlled), conservation of precious samples and reagents, and limited assay cross-reactivity due to the parallel nature of the multiplexing platform. In addition, the microfluidic platform has been shown to be more sensitive than traditional ELISA, xMAP, or flow cytometry platforms, allowing for detection of smaller concentrations of biomarkers/analytes in a given sample.

In some embodiments, assessing biomarker expression is assessed via nucleic acid detection. For example, in alternative embodiments for detecting protein expression, one may assay for gene transcription. For example, an indirect method for detecting protein expression is to detect mRNA transcripts from which the proteins are made. The following is a discussion of such methods, which are applicable particularly to calcyclin, calpactin I light chain, astrocytic phosphoprotein PEA-15 and tubulin-specific chaperone A in the context of the present disclosure.

In some embodiments, assessing biomarker expression may comprise amplification of nucleic acids. Since many mRNAs are present in relatively low abundance, nucleic acid amplification greatly enhances the ability to assess expression. The general concept is that nucleic acids can be amplified using paired primers flanking the region of interest. The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to selected genes are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemilluminescence, radioactive scintigraphy of incorporated radiolabel or fluorescent label or even via a system using electrical and/or thermal impulse signals.

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known. Alternative methods for reverse transcription utilize thermostable DNA polymerases, for example, as described in WO 90/07641. Polymerase chain reaction methodologies are well known in the art. Representative methods of RT-PCR are described in U.S. Pat. No. 5,882,864.

Whereas standard PCR usually uses one pair of primers to amplify a specific sequence, multiplex-PCR (MPCR) uses multiple pairs of primers to amplify many sequences simultaneously. The presence of many PCR primers in a single tube could cause many problems, such as the increased formation of misprimed PCR products and “primer dimers,” the amplification discrimination of longer DNA fragment and so on. Normally, MPCR buffers contain a Taq Polymerase additive, which decreases the competition among amplicons and the amplification discrimination of longer DNA fragment during MPCR. MPCR products can further be hybridized with gene-specific probe for verification. Theoretically, one should be able to use as many as primers as necessary. However, due to side effects (primer dimers, misprimed PCR products, etc.) caused during MPCR, there is a limit (less than 20) to the number of primers that can be used in a MPCR reaction.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 4,912,148, may also be used.

Alternative methods for amplification of target nucleic acid sequences that may be used in the practice of the present disclosure are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which are incorporated herein by reference in its entirety.

Qbeta Replicase, described in PCT Application No. PCT/US87/00880, may also be used as an amplification method in the present disclosure. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site, may also be useful in the amplification of nucleic acids in the present disclosure. Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR. European Application No. 329 822 disclose a nucleic acid amplification process involving cyclically synthesizing single-stranded RNA (“ssRNA”), ssDNA, and double-stranded DNA (dsDNA), which may be used in accordance with the present disclosure.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR.”

In some embodiments, assessing biomarker expression may comprise detection of nucleic acids. Following any amplification, it may be desirable to separate the amplification product from the template and/or the excess primer. In one embodiment, amplification products are separated by agarose, agarose-acrylamide or polyacrylamide gel electrophoresis using standard methods. Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the presently-disclosed subject matter, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present disclosure.

Other methods of nucleic acid detection that may be used in the practice of the instantly-disclosed subject matter are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

In some embodiments, assessing biomarker expression may comprise a nucleic acid array. Microarrays comprise a plurality of polymeric molecules spatially distributed over, and stably associated with, the surface of a substantially planar substrate, e.g., biochips. Microarrays of polynucleotides have been developed and find use in a variety of applications, such as screening and DNA sequencing. One area in particular in which microarrays find use is in gene expression analysis.

In gene expression analysis with microarrays, an array of “probe” oligonucleotides is contacted with a nucleic acid sample of interest, i.e., target, such as polyA mRNA from a particular tissue type. Contact is carried out under hybridization conditions and unbound nucleic acid is then removed. The resultant pattern of hybridized nucleic acid provides information regarding the genetic profile of the sample tested. Methodologies of gene expression analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phophorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,2613, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed, and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

In some embodiments, assessing biomarker expression may comprise RNA sequencing. RNA-seq (RNA Sequencing), also called Whole Transcriptome Shotgun Sequencing (WTSS), is a technology that utilizes the capabilities of next-generation sequencing to reveal a snapshot of RNA presence and quantity from a genome at a given moment in time.

The transcriptome of a cell is dynamic; it continually changes as opposed to a static genome. The recent developments of Next-Generation Sequencing (NGS) allow for increased base coverage of a DNA sequence, as well as higher sample throughput. This facilitates sequencing of the RNA transcripts in a cell, providing the ability to look at alternative gene spliced transcripts, post-transcriptional changes, gene fusion, mutations/SNPs and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries, Ongoing RNA-Seq research includes observing cellular pathway alterations during infection, and gene expression level changes in cancer studies. Prior to NGS, transcriptomics and gene expression studies were previously done with expression microarrays, which contain thousands of DNA sequences that probe for a match in the target sequence, making available a profile of all transcripts being expressed. This was later done with Serial Analysis of Gene Expression (SAGE).

One deficiency with microarrays that makes RNA-Seq more attractive has been limited coverage; such arrays target the identification of known common alleles that represent approximately 500,000 to 2,000,000 SNPs of the more than 10,000,000 in the genome. As such, libraries aren't usually available to detect and evaluate rare allele variant transcripts, and the arrays are only as good as the SNP databases they're designed from, so they have limited application for research purposes. Many cancers for example are caused by rare <1% mutations and would go undetected. However, arrays still have a place for targeted identification of already known common allele variants, making them ideal for regulatory-body approved diagnostics such as cystic fibrosis.

In some embodiments, assessing biomarker expression may comprise utilizing a RNA “Poly(A)” Library. Creation of a sequence library can change from platform to platform in high throughput sequencing, where each has several kits designed to build different types of libraries and adapting the resulting sequences to the specific requirements of their instruments. However, due to the nature of the template being analyzed, there are commonalities within each technology. Frequently, in mRNA analysis the 3′ polyadenylated (poly(A)) tail is targeted in order to ensure that coding RNA is separated from noncoding RNA. This can be accomplished simply with poly (T) oligos covalently attached to a given substrate. Presently many studies utilize magnetic beads for this step. The Protocol Online website provides a list of several protocols relating to mRNA isolation.

Studies including portions of the transcriptome outside poly(A) RNAs have shown that when using poly(T) magnetic beads, the flow-through RNA (non-poly(A) RNA) can yield important noncoding RNA gene discovery which would have otherwise gone unnoticed. Also, since ribosomal RNA represents over 90% of the RNA within a given cell, studies have shown that its removal via probe hybridization increases the capacity to retrieve data from the remaining portion of the transcriptome.

The next step is reverse transcription. Due to the 5′ bias of randomly primed-reverse transcription as well as secondary structures influencing primer binding sites, hydrolysis of RNA into 200-300 nucleotides prior to reverse transcription reduces both problems simultaneously. However, there are trade-offs with this method where although the overall body of the transcripts are efficiently converted to DNA, the 5′ and 3′ ends are less so. Depending on the aim of the study, researchers may choose to apply or ignore this step.

Once the cDNA is synthesized it can be further fragmented to reach the desired fragment length of the sequencing system.

Small RNA/Non-coding RNA sequencing. When sequencing RNA other than mRNA the library preparation is modified. The cellular RNA is selected based on the desired size range. For small RNA targets, such as miRNA, the RNA is isolated through size selection. This can be performed with a size exclusion gel, through size selection magnetic beads, or with a commercially developed kit. Once isolated, linkers are added to the 3′ and 5′ end then purified. The final step is cDNA generation through reverse transcription.

Additionally or alternatively, in some embodiments, assessing biomarker expression may comprise utilizing direct RNA sequencing. As converting RNA into cDNA using reverse transcriptase has been shown to introduce biases and artifacts that may interfere with both the proper characterization and quantification of transcripts, single molecule Direct RNA Sequencing (DRSTM) technology is currently under development by Helicos. DRSTM sequences RNA molecules directly in a massively-parallel manner without RNA conversion to cDNA or other biasing sample manipulations such as ligation and amplification.

Two different assembly methods are used for producing a transcriptome from raw sequence reads: de-novo and genome-guided. The first approach does not rely on the presence of a reference genome in order to reconstruct the nucleotide sequence. Due to the small size of the short reads de novo assembly may be difficult though some software does exist (Velvet (algorithm), Oases, and Trinity to mention a few), as there cannot be large overlaps between each read needed to easily reconstruct the original sequences. The deep coverage also makes the computing power to track all the possible alignments prohibitive. This deficit can improved using longer sequences obtained from the same sample using other techniques such as Sanger sequencing, and using larger reads as a “skeleton” or a “template” to help assemble reads in difficult regions (e.g., regions with repetitive sequences).

An “easier” and relatively computationally cheaper approach is that of aligning the millions of reads to a “reference genome.” There are many tools available for aligning genomic reads to a reference genome (sequence alignment tools), however, special attention is needed when alignment of a transcriptome to a genome, mainly when dealing with genes having intronic regions. Several software packages exist for short read alignment, and recently specialized algorithms for transcriptome alignment have been developed, e.g. Bowtie for RNA-seq short read alignment, TopHat for aligning reads to a reference genome to discover splice sites, Cufflinks to assemble the transcripts and compare/merge them with others, or FANSe. These tools can also be combined to form a comprehensive system.

Although numerous solutions to the assembly quest have been proposed, there is still room for improvement given the resulting variability of the approaches. A group from the Center for Computational Biology at the East China Normal University in Shanghai compared different de novo and genome-guided approaches for RNA-Seq assembly. They noted that, although most of the problems can be solved using graph theory approaches, there is still a consistent level of variability in all of them. Some algorithms outperformed the common standards for some species while still struggling for others. The authors suggest that the “most reliable” assembly could be then obtained by combining different approaches. Interestingly, these results are consistent with NGS-genome data obtained in a recent contest called Assemblathon where 21 contestants analyzed sequencing data from three different vertebrates (fish, snake and bird) and handed in a total of 43 assemblies. Using a metric made of 100 different measures for each assembly, the reviewers concluded that 1) assembly quality can vary a lot depending on which metric is used and 2) assemblies that scored well in one species didn't really perform well in the other species.

As discussed above, sequence libraries are created by extracting mRNA using its poly(A) tail, which is added to the mRNA molecule post-transcriptionally and thus splicing has taken place. Therefore, the created library and the short reads obtained cannot come from intronic sequences, so library reads spanning the junction of two or more exons will not align to the genome.

A possible method to work around this is to try to align the unaligned short reads using a proxy genome generated with known exonic sequences. This need not cover whole exons, only enough so that the short reads can match on both sides of the exon-exon junction with minimum overlap. Some experimental protocols allow the production of strand specific reads.

The characterization of gene expression in cells via measurement of mRNA levels has long been of interest to researchers, both in terms of which genes are expressed in what tissues, and at what levels. Even though it has been shown that due to other post transcriptional gene regulation events (such as RNA interference) there is not necessarily always a strong correlation between the abundance of mRNA and the related proteins, measuring mRNA concentration levels is still a useful tool in determining how the transcriptional machinery of the cell is affected in the presence of external signals (e.g., drug treatment), or how cells differ between a healthy state and a diseased state.

Expression can be deduced via RNA-seq to the extent at which a sequence is retrieved. Transcriptome studies in yeast show that in this experimental setting, a four-fold coverage is required for amplicons to be classified and characterized as an expressed gene. When the transcriptome is fragmented prior to cDNA synthesis, the number of reads corresponding to the particular exon normalized by its length in vivo yields gene expression levels which correlate with those obtained through qPCR.

The only way to be absolutely sure of the individual's mutations is to compare the transcriptome sequences to the germline DNA sequence. This enables the distinction of homozygous genes versus skewed expression of one of the alleles and it can also provide information about genes that were not expressed in the transcriptomic experiment. An R-based statistical package known as CummeRbund can be used to generate expression comparison charts for visual analysis.

In some embodiments, the LFPI may be determined by a Lupus Flare Prediction Algorithm. For example, in some embodiments, the Lupus Flare Prediction Algorithm may comprise a series of steps to determine the LFPI for the patient.

In some embodiments, the LFPI may be determined as follows:
1. Soluble mediator data for each analyte (Pre-flare and Pre-nonflare) are log transformed and standardized by subtracting a mean concentration of log-transformed analyte, then dividing by the standard deviation [SD] of log-transformed analyte;
2. Spearman r correlation values for each analyte (Pre-flare and Pre-nonflare samples) are determined between plasma sample soluble mediator concentration at baseline vs. hybrid (hSLEDAI) (SELENA-SLEDAI with proteinuria as defined by SLEDAI-2K) score at follow-up visit (time of concurrent clinical disease flare or nonflare);
3. Log-transformed, standardized soluble mediator data from Step 1 are then weighted (e.g., multiplied) by Spearman r from Step 2 for each analyte; and
4. The LFPI subscores for analytes informing the LFPI are summed to give a total LFPI score.

In some embodiments, the biomarkers utilized may comprise or consist of OPN, MCP-1/CCL2, MCP-3/CCL7, IL-17A, TNFRII, TNFRI, IL-4, IL-5, BLyS, TNF-α, and IL-7. In some embodiments, these eleven (11) biomarkers utilized in the Lupus Flare Prediction Algorithm, as disclosed herein, may be effective to predict flares, for example, before clinical symptoms are reported, with accuracy at least as good as alternative algorithms necessitating substantially more biomarkers, for example, 25 or more biomarkers, 30 or more biomarkers, 35 or more biomarkers, or 40 or more biomarkers. Not intending to be bound by theory, because of the necessity for data from fewer biomarkers, the LFPI may be more viable than other algorithms.

In some embodiments, the LFPI score calculated according to the methods disclosed herein may be utilized to, based upon the LFPI, distinguish a lupus patients in a pre-flare state (positive risk score) from a pre-nonflare state (negative risk score).

### Treating SLE

Advantages accruing to the presently-disclosed subject matterinclude earlier intervention, when the symptoms of a flare have not appeared. Thus, the present subject matter contemplates the treatment of SLE using standard therapeutic approaches where indicated. Although decisions related to the treatment of SLE will be made by a physician, in general, the treatment of SLE involves treating elevated disease activity and trying to minimize the organ damage that can be associated with this increased inflammation and increased immune complex formation/deposition/complement activation. Foundational treatment can include corticosteroids and anti-malarial drugs. Certain types of lupus nephritis such as diffuse proliferative glomerulonephritis require bouts of cytotoxic drugs. These drugs include, most commonly, cyclophosphamide and mycophenolate. Hydroxychloroquine (HCQ) was approved by the FDA for lupus in 1955. Some drugs approved for other diseases are used for SLE “off-label.” Belimumab (Benlysta) has been approved as a treatment for elevated disease activity seen in autoantibody-positive lupus patients.

Due to the variety of symptoms and organ system involvement with SLE, its severity in an individual must be assessed in order to successfully treat SLE. Mild or remittent disease may, sometimes, be safely left minimally treated with hydroxychloroquine alone. If required, nonsteroidal anti-inflammatory drugs and low dose steroids may also be used. Hydroxychloroquine (HCQ) is an FDA-approved antimalarial used for constitutional, cutaneous, and articular manifestations. Hydroxychloroquine has relatively few side effects, and there is evidence that it improves survival among people who have SLE and stopping HCQ in stable SLE patients led to increased disease flares in Canadian lupus patients. Disease-modifying antirheumatic drugs (DMARDs) are oftentimes used off-label in SLE to decrease disease activity and lower the need for steroid use. DMARDs commonly in use are methotrexate and azathioprine. In more severe cases, medications that aggressively suppress the immune system (primarily high-dose corticosteroids and major immunosuppressants) are used to control the disease and prevent damage. Cyclophosphamide is used for severe glomerulonephritis, as well as other life-threatening or organ-damaging complications, such as vasculitis and lupus cerebritis. Mycophenolate mofetil is also used for treatment of lupus nephritis, but it is not FDA-approved for this indication.

Depending on the dosage, people who require steroids may develop Cushing's symptoms of truncal obesity, purple striae, buffalo hump and other associated symptoms. These may subside if and when the large initial dosage is reduced, but long-term use of even low doses can cause elevated blood pressure, glucose intolerance (including metabolic syndrome and/or diabetes), osteoporosis, insomnia, avascular necrosis and cataracts.

Numerous new immunosuppressive drugs are being actively tested for SLE. Rather than suppressing the immune system nonspecifically, as corticosteroids do, they target the responses of individual types of immune cells. Belimumab, or a humanized monoclonal antibody against B-lymphocyte stimulating factor (BlyS or BAFF), is FDA approved for lupus treatment and decreased SLE disease activity, especially in patients with baseline elevated disease activity and the presence of autoantibodies. Addition drugs, such as abatacept, epratuzimab, etanercept and others, are actively being studied in SLE patients and some of these drugs are already FDA-approved for treatment of rheumatoid arthritis or other disorders. Since a large percentage of people with SLE suffer from varying amounts of chronic pain, stronger prescription analgesics (pain killers) may be used if over-the-counter drugs (mainly nonsteroidal anti-inflammatory drugs) do not provide effective relief. Potent NSAIDs such as indomethacin and diclofenac are relatively contraindicated for patients with SLE because they increase the risk of kidney failure and heart failure.

Moderate pain is typically treated with mild prescription opiates such as dextropropoxyphene and co-codamol. Moderate to severe chronic pain is treated with stronger opioids, such as hydrocodone or longer-acting continuous-release opioids, such as oxycodone, MS Contin, or methadone. The fentanyl duragesic transdermal patch is also a widely used treatment option for the chronic pain caused by complications because of its long-acting timed release and ease of use. When opioids are used for prolonged periods, drug tolerance, chemical dependency, and addiction may occur. Opiate addiction is not typically a concern, since the condition is not likely to ever completely disappear. Thus, lifelong treatment with opioids is fairly common for chronic pain symptoms, accompanied by periodic titration that is typical of any long-term opioid regimen.

Intravenous immunoglobulins may be used to control SLE with organ involvement, or vasculitis. It is believed that they reduce antibody production or promote the clearance of immune complexes from the body, even though their mechanism of action is not well-understood. Unlike immunosuppressives and corticosteroids, IVIGs do not suppress the immune system, so there is less risk of serious infections with these drugs.

Avoiding sunlight is the primary change to the lifestyle of SLE sufferers, as sunlight is known to exacerbate the disease, as is the debilitating effect of intense fatigue. These two problems can lead to patients becoming housebound for long periods of time. Drugs unrelated to SLE should be prescribed only when known not to exacerbate the disease. Occupational exposure to silica, pesticides and mercury can also make the disease worsen.

Renal transplants are the treatment of choice for end-stage renal disease, which is one of the complications of lupus nephritis, but the recurrence of the full disease in the transplanted kidney is common in up to 30% of patients.

Antiphospholipid syndrome is also related to the onset of neural lupus symptoms in the brain. In this form of the disease the cause is very different from lupus: thromboses (blood clots or “sticky blood”) form in blood vessels, which prove to be fatal if they move within the blood stream. If the thromboses migrate to the brain, they can potentially cause a stroke by blocking the blood supply to the brain. If this disorder is suspected in patients, brain scans are usually required for early detection. These scans can show localized areas of the brain where blood supply has not been adequate. The treatment plan for these patients requires anticoagulation. Often, low-dose aspirin is prescribed for this purpose, although for cases involving thrombosis anticoagulants such as warfarin are used.

### Pharmaceutical Formulations and Delivery

Where therapeutic applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the vector to cells, dissolved or dispersed in a pharmaceutically acceptable carrier or aqueous medium. Such compositions also are referred to as inocula. The phrases “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the presently-disclosed subject matter, its use in therapeutic compositions is contemplated. Supplementary active ingredients also can be incorporated into the compositions.

The active compositions of the presently-disclosed subject matter may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intraperitoneal, or intravenous injection. Such compositions would normally be administered as pharmaceutically acceptable compositions.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present disclosure may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### Kits

For use in the applications described herein, kits are also within the scope of this disclosure. Such kits can comprise a carrier, package or container that is compartmentalized to receive one or more containers such as vials, tubes, and the like, each of the container(s) comprising one of the separate elements to be used in the method, in particular, a Bright inhibitor. The kit will typically comprise the container described above and one or more other containers comprising materials desirable from a commercial end user standpoint, including buffers, diluents, filters, and package inserts with instructions for use. In addition, a label can be provided on the container to indicate that the composition is used for a specific therapeutic application, and can also indicate directions for either in vivo or in vitro use, such as those described above. Directions and or other information can also be included on an insert which is included with the kit. In particular, kits according to the present disclosure contemplate the assemblage of agents for assessing levels of the biomarkers discussed above along with one or more of an SLE therapeutic and/or a reagent for assessing antinuclear antibody (ANA) testing and/or anti-extractable nuclear antigen (anti-ENA), as well as controls for assessing the same.

### EXAMPLES

The following examples are included to further illustrate various aspects of this disclosure. It should be appreciated by those of skill in the art that the techniques disclosed in the examples which follow represent techniques and/or compositions can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosed subject matter.

### Example 1

Example 1 details a study demonstrating the efficacy of the methods disclosed herein.

Study Population: The levels of 38 biomarkers representing SLE-associated inflammatory and regulatory soluble mediators were assessed in prospective study samples from 106 patients longitudinally. Patients were sampled at Baseline, 3-, 6-, 9-, and 12-months with blood samples for plasma isolation and clinical/medication data collected at each visit. The characteristics of the SLE patient population analyzed in these studies is shown in Table 1.

TABLE 1
Baseline SLE Patient Parameters (n = 106)

| | | |
|---|---|---|
| Female (n, %) | 97 | (92%) |
| Race (n, %) | | |
| European American | 63 | (60%) |
| African American | 21 | (20%) |
| Native American | 11 | (10%) |
| Other^{a} | 11 | (10%) |
| Age (mean, SD) | 44 | (14) |
| hSLEDAI^{b} (mean, SD, range) | 4.1 | (3.6 [0-17]) |
| Clinical Disease activity (n, %) | | |
| Low (hSLEDAI 0-3) | 48 | (45%) |
| Active (hSLEDAI 4-6) | 39 | (37%) |
| High (hSLEDAI ≥8) | 19 | (18%) |
| Organ System Manifestations^{c} (n, %) | | |
| Arthritis | 25 | (24%) |
| Renal | 16 | (15%) |
| Mucocutaneous | 47 | (44%) |
| Serositis | 8 | (8%) |
| Immunologic | 43 | (41%) |
| Hematologic | 9 | (8%) |
| Medications^{d} (n %) | | |
| Steroid | 58 | (55%) |
| Hydroxychloroquine | 80 | (75%) |
| Immunosuppressants | 36 | (34%) |
| Major Immunosupressants | 25 | (24%) |

^{a}Other = Asian, Hispanic, Multi-race, and Unknown
^{b}hSLEDAI = SELENA-SLEDAI + proteinuria defined by SLEDAI-2K
^{c}No CNS, Vasculitis, Myositis, or Fever at BL visits
^{d}Immunosuppressants = azathioprine, methotrexate, sirolimus, tacrolimus; Major Immunosuppressants = mycophenolate mofetil, cyclophosphamide

Clinical Data and Flare Determination: Disease activity and flare were determined at three month/12 week intervals by hybrid (hSLEDAI) clinical disease activity instrument for SLE (SELENA-SLEDAI with proteinuria as defined by SLEDAI-2K). Pre-flare visits (three months prior to hSLEDAI clinical disease flare) and Pre-nonflare visits (comparable three-month period between baseline and follow-up visits in which no hSLEDAI defined clinical disease flare took place) were subsequently selected.

Biomarker Analysis: Undiluted plasma samples were procured from appropriately consented SLE patients described above, promptly processed, divided into aliquots, and frozen at −80° C. Quantitative levels of plasma cytokines were determined for 38 candidate biomarkers listed in Table 2. The majority of the 38 biomarkers assessed represents a subset of a previously-investigated 52 biomarker panel.

Soluble mediator concentrations on freshly thawed sample aliquots for each Pre-flare and Pre-nonflare sample were determined using the ProteinSimple/Bio-techne Ella/Simple Plex™ (Ella) automated microfluidic immunoassay platform. Ella is a highly robust cartridge-based immunoassay platform. Known quality control samples (3-levels) are included on each cartridge to assess inter- and intra-assay variance. Published mean inter-assay coefficient of variation (CV) of the Ella platform for cytokine detection has previously been shown to be <10% and our observed CVs for the analytes under investigation are <10%. Following data collection, biomarker concentrations (pg/ml) are interpolated from a built-in six-point standard curve using the on-board Simple Plex Explorer software which employs a 5-PL curve fit. Mean concentration values are obtained for each of the biomarkers and utilized for statistical analyses.

TABLE 2
Pathways of Candidate Biomarkers Examined in Study

Innate
IL-1α
IL-1β
IL-1RA
IFN-α 2
Regulatory
IL-10
TGF-β (native)
TGF-β (total)
Other
OPN
Resistin
SCF
Th1-type
IFN-γ
IL-2
IL-2Rα
IL-12 (p70)
Th2-like
IL-4
IL-5
IL-13
Th17-like
IL-6
IL-17A
Chemokine/Adhesion
IL-8/CXCL8
MIG/CXCL9
IP-10/CXCL10
MCP-1/CCL2
MIP-1α/CCL3
MIP-1β/CCL4
RANTES/CCL5
MCP-3/CCL7
ICAM-1
VCAM-1
Metalloproteinase
MMP-9
TIMP-1
TNF Superfamily
BAFF/BLyS
Fas
TNFRI (p55)
TNFRII (p75)
TRAIL
TNF-α
Homeostasis
IL-7
IL-15

Statistical Analyses. Random forest statistical analyses were performed with log-transformed, standardized biomarker data to rank variables by their ability to distinguish Pre-flare from Pre-nonflare samples (Table 3). The random forest variable importance ranking was obtained from 2000 repeats; variable importance and cumulative importance are shown below. In these analyses, OPN is ranked first or most important among the biomarkers.

TABLE 3
Random Forest Variable Importance (2000X)

| | | | Importance |
|---|---|---|---|
| Variable | Order | Importance | Cumulative |
| OPN | 1 | 0.82 | 0.82 |
| MCP_1_per_CCL2 | 2 | 0.75 | 1.57 |
| MCP_3_per_CCL7 | 3 | 0.73 | 2.30 |
| IL_17A | 4 | 0.66 | 2.97 |
| TNFRII | 5 | 0.64 | 3.60 |
| TNFRI | 6 | 0.60 | 4.20 |
| IL_4 | 7 | 0.56 | 4.75 |
| IL_5 | 8 | 0.52 | 5.27 |
| MMP_9 | 9 | 0.49 | 5.76 |
| IL_8_per_CXCL8 | 10 | 0.48 | 6.24 |
| TNF_a | 11 | 0.47 | 6.71 |
| IL_7 | 12 | 0.47 | 7.17 |
| IL_10 | 13 | 0.47 | 7.64 |
| IL_2Ra | 14 | 0.45 | 8.09 |
| IFN_a | 15 | 0.43 | 8.53 |
| Age_at_Exam | 16 | 0.42 | 8.95 |
| IP_10_per_CXCL10 | 17 | 0.42 | 9.37 |
| BLyS | 18 | 0.41 | 9.78 |
| IL_1RA | 19 | 0.41 | 10.19 |
| MIG_per_CXCL9 | 20 | 0.40 | 10.59 |
| IL_6 | 21 | 0.40 | 10.99 |
| TRAIL | 22 | 0.39 | 11.38 |
| Total_TGF_b1 | 23 | 0.39 | 11.76 |
| Resistin | 24 | 0.37 | 12.14 |
| IL_1b | 25 | 0.37 | 12.51 |
| IL_12p70 | 26 | 0.37 | 12.88 |
| ICAM_1 | 27 | 0.36 | 13.24 |
| IFN_g | 28 | 0.36 | 13.60 |
| TIMP_1 | 29 | 0.36 | 13.96 |
| IL_13 | 30 | 0.36 | 14.32 |
| IL_15 | 31 | 0.35 | 14.67 |
| VCAM_1 | 32 | 0.34 | 15.01 |
| RANTES_per_CCL5 | 33 | 0.32 | 15.34 |
| SCF | 34 | 0.31 | 15.65 |
| Fas | 35 | 0.30 | 15.95 |
| MIP_1a_per_CCL3 | 36 | 0.28 | 16.23 |
| MIP_1b_per_CCL4 | 37 | 0.27 | 16.50 |
| Native_TGF_b1 | 38 | 0.25 | 16.75 |
| Steroid | 39 | 0.18 | 16.93 |
| Immunologic | 40 | 0.11 | 17.05 |
| Arthritis | 41 | 0.10 | 17.15 |
| Mucocutaneous | 42 | 0.09 | 17.24 |
| Anti_Malarial | 43 | 0.08 | 17.33 |
| Race | 44 | 0.07 | 17.40 |
| Immunosuppressant | 45 | 0.06 | 17.46 |
| Major_Immunosuppressant | 46 | 0.03 | 17.49 |
| Gender | 47 | 0.01 | 17.51 |
| Hematologic | 48 | 0.00 | 17.51 |

The LFPI is the summation of LFPI calculations for each biomarker informing the algorithm as follows:
1. Soluble mediator data (Pre-flare and Pre-nonflare) were log transformed and standardized (subtract mean concentration of log-transformed analyte, then divided by standard deviation [SD] of log-transformed analyte);
2. Spearman r correlation values for each analyte (Pre-flare and Pre-nonflare samples) were determined between plasma sample soluble mediator concentration at baseline vs. hSLEDAI score at follow-up visit (time of concurrent clinical disease flare or nonflare);
3. Log-transformed, standardized soluble mediator data (Step 1) were then weighted (multiplied) by Spearman r (Step 2) for each biomarker;
4. The LFPI subscores for biomarkers informing the LFPI were summed to give a total LFPI score.

After logistic regression cross-validation to determine optimal analyte groupings by forward selection (based on random forest variable importance), the best performing analyte groupings (Table 4) were applied to the LFPI calculation. A confusion matrix was completed for each iteration to determine the number of analytes that maximize LFPI performance (based on cutoff of “0” for Pre-flare [positive values] vs. Pre-nonflare [negative values], as well as a cutoff based on the LFPI value that maximizes sensitivity and specificity [Youden index]). The top performing LFPI iterations contained 11 to 13 biomarkers out of the 38 total candidates (Table 4).

TABLE 4
LFPI Biomarkers Grouped Based on Random Forest
Variable Importance and Multivariate Analyses.
LFPI (G) Remove IL-8

| | | | |
|---|---|---|---|
| # Analytes | 1:10 | 1:2 (multi) | 1:2(single) |
| | OPN | MCP-1/CCL2 | MCP-3/CCL7 |
| | TNFRI | IL-17A | |
| | TNFRII | IL-4 | |
| | BLyS | IL-5 | |
| | | TNF-α | |
| LFPI 11G | | IL-7 | |

LFPI (A)

| | | | | |
|---|---|---|---|---|
| # Analytes | 1:100 | 1:10 | 1:2 (multi) | 1:2 (single) |
| | MMP-9 | OPN | MCP-1/CCL2 | MCP-3/CCL7 |
| | | TNFRI | IL-17A | IL-8/CXCL8 |
| | | TNFRII | IL-4 | |
| | | | IL-5 | |
| | | | TNF-α | |
| LFPI 12A | | | IL-7 | |

LFPI (B) Substitute Resistin for MMP-9

| | | | |
|---|---|---|---|
| # Analytes | 1:10 | 1:2 (multi) | 1:2 (single) |
| | OPN | MCP-1/CCL2 | MCP-3/CCL7 |
| | TNFRI | IL-17A | IL-8/CXCL8 |
| | TNFRII | IL-4 | |
| | Resistin | IL-5 | |
| | | TNF-α | |
| LFPI 12B | | IL-7 | |

LFPI (E) = LFPI (A) + BLyS

| | | | | |
|---|---|---|---|---|
| # Analytes | 1:100 | 1:10 | 1:2 (multi) | 1:2 (single) |
| | MMP-9 | OPN | MCP-1/CCL2 | MCP-3/CCL7 |
| | | TNFRI | IL-17A | IL-8/CXCL8 |
| | | TNFRII | IL-4 | |
| | | BLyS | IL-5 | |
| | | | TNF-α | |
| LFPI 13E | | | IL-7 | |

LFPI (F) = Substitute BlyS for MMP-9

| | | | |
|---|---|---|---|
| # Analytes | 1:10 | 1:2 (multi) | 1:2 (single) |
| | OPN | MCP-1/CCL2 | MCP-3/CCL7 |
| | TNFRI | IL-17A | IL-8/CXCL8 |
| | TNFRII | IL-4 | |
| | BLyS | IL-5 | |
| | | TNF-α | |
| LFPI 12F | | IL-7 | |

These top five LFPI iterations performed similarly in discriminating Pre-flare from Pre-nonflare samples as shown below in Table 5.

TABLE 5
Top Performing LFPI Biomarker Groupings

| | | LFPI Levels | | | | | | | | | | | | |
|---|---|---|---|---|---|---|---|---|---|---|---|---|---|---|
| Overall | | Pre-Flare | | Pre-Nonflare | | | | | | Correlation with hSLEDAI @ FU | | | | |
| Rank | | (n = 46) | | (n = 53) | | | AUC | | | Spearman | | | | |
| Order | LFPI | Mean | SEM | Mean | SEM | p-value | AUC | 95% CI | p-value | r | 95% CI | p-value | | |
| 1 | LFPI 13E | 3.36 | 1.05 | −2.82 | 0.84 | <0.0001 | 0.756 | 0.660- | <0.0001 | 0.440 | 0.260 to | <0.0001 | | |
| | | | | | | | | 0.851 | | | 0.590 | | | |
| 2 | LFPI 12A | 3.18 | 0.98 | −2.67 | 0.80 | <0.0001 | 0.754 | 0.659- | <0.0001 | 0.439 | 0.259 to | <0.0001 | | |
| | | | | | | | | 0.849 | | | 0.590 | | | |
| 3 | LFPI 11G | 3.19 | 1.00 | −2.66 | 0.85 | <0.0001 | 0.752 | 0.658- | <0.0001 | 0.434 | 0.254 to | <0.0001 | | |
| | | | | | | | | 0.847 | | | 0.586 | | | |
| 4 | LFPI 12F | 3.23 | 1.03 | −2.69 | 0.86 | <0.0001 | 0.747 | 0.652- | <0.0001 | 0.415 | 0.231 to | <0.0001 | | |
| | | | | | | | | 0.843 | | | 0.570 | | | |
| 5 | LFPI 12B | 3.09 | 0.96 | −2.57 | 0.82 | <0.0001 | 0.746 | 0.650- | <0.0001 | 0.412 | 0.229 to | <0.0001 | | |
| | | | | | | | | 0.842 | | | 0.568 | | | |
| Overall | | Positive/Negative Cutoff = 0 | | | | | | Positive/Negative Cutoff Based on Youden Index | | | | | | |
| Rank | | Odds Ratio | Sensi- | Speci- | | | | | Odds Ratio | Sensi- | Speci- | | | |
| Order | LFPI | (95% CI) | tivity | ficity | PPV | NPV | p-value | Cutoff | (95% CI) | tivity | ficity | PPV | NPV | p-value |
| 1 | LFPI 13E | 4.8 (2.0-11) | 0.67 | 0.70 | 0.65 | 0.72 | 0.0003 | 0.1542 | 7.5 (3.0-18) | 0.60 | 0.83 | 0.75 | 0.71 | <0.0001 |
| 2 | LFPI 12A | 4.3 (1.8-9.4) | 0.64 | 0.70 | 0.64 | 0.70 | 0.0006 | −0.2788 | 8.1 (2.9-22) | 0.87 | 0.56 | 0.62 | 0.83 | <0.0001 |
| 3 | LFPI 11G | 4.8 (2.0-11) | 0.67 | 0.70 | 0.65 | 0.72 | 0.0003 | −0.2683 | 6.8 (2.6-17) | 0.84 | 0.56 | 0.61 | 0.81 | <0.0001 |
| 4 | LFPI 12F | 4.8 (2.0-11) | 0.67 | 0.70 | 0.65 | 0.72 | 0.0003 | 0.1368 | 6.6 (2.7-15) | 0.60 | 0.81 | 0.73 | 0.71 | <0.0001 |
| 5 | LFPI 12B | 4.0 (1.6-8.8) | 0.67 | 0.67 | 0.64 | 0.69 | 0.0020 | 0.1810 | 5.9 (2.5-15) | 0.60 | 0.80 | 0.71 | 0.71 | <0.0001 |

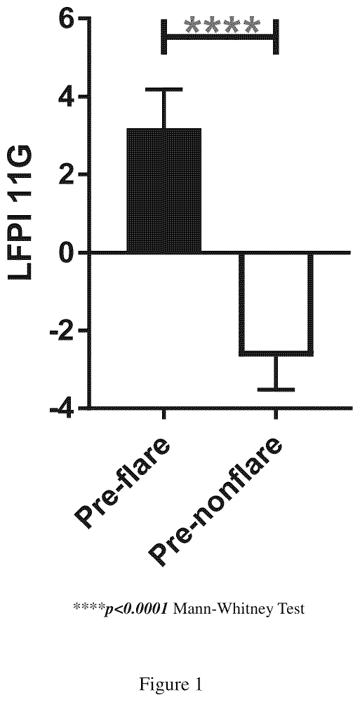

Following internal cross validation, the LFPI 11G was found to perform optimally with an AUC of 0.76 (0.75-0.77). FIG. 1 shows additional performance results for 11G. The calculated 11G LFPI significantly distinguishes lupus patients in a pre-flare state (positive risk score) vs. a pre-nonflare state (negative risk score).

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the subject matter defined by the appended claims.

What is claimed is:

1. A method for determining whether a Systemic lupus erythematosus (SLE) patient is undergoing a pre-flare event, the method comprising:
obtaining a blood, serum, plasma, or saliva sample from the SLE patient; and
assessing a level of expression for each of a plurality of biomarkers, the plurality of biomarkers comprising Osteopontin (OPN), monocyte chemotactic protein-1 (MCP-1/CCL2), monocyte-specific chemokine 3 (MCP-3/CCL7), interleukin (IL-17A), tumor necrosis factor II (TNFRII), tumor necrosis factor receptor I (TNFRI), interleukin-4 (IL-4), interleukin-5 (IL-5), tumor necrosis factor-alpha (TNF-α), and interleukin-7 (IL-7).

2. The method of claim 1, further comprising determining a Lupus Flare Risk Prediction Index (LFPI) for the SLE patient based upon the level of expression for each of a plurality of biomarkers.

3. The method of claim 2, wherein determining the LFPI for the SLE patient based upon the level of expression for each of a plurality of biomarkers comprises log transforming data for each biomarker and standardizing the log-transformed mediator data, then dividing by a standard deviation [SD] of the log-transformed mediator data.

4. The method of claim 3, wherein determining the LFPI for the SLE patient based upon the level of expression for each of a plurality of biomarkers further comprises determining a Spearman r correlation value for each biomarker between plasma sample soluble mediator concentration at a baseline compared to hybrid SLEDAI (hSLEDAI) score.

5. The method of claim 4, wherein determining the LFPI for the SLE patient based upon the level of expression for each of a plurality of biomarkers further comprises weighting the log-transformed, standardized data from by multiplying by the Spearman r for each biomarker to yield a subscore for each biomarker.

6. The method of claim 3, wherein determining the LFPI for the SLE patient based upon the level of expression for each of a plurality of biomarkers further comprises summing the subscores for the plurality of biomarkers to give the LFPI for the SLE patient.

7. The method of claim 2, further comprising, based upon the LFPI, determining whether the SLE patient is undergoing a pre-flare event.

8. The method of claim 2, wherein the plurality of biomarkers further comprises one or more of B lymphocyte stimulator (BLyS), matrix metallopeptidase ((MMP-9), Resistin, interleukin-8 (IL-8/CXCL8).

9. The method of claim 8, wherein the plurality of biomarkers consists of OPN, MCP-1/CCL2, MCP-3/CCL7, IL-17A, TNFRII, TNFRI, IL-4, IL-5, BLyS, TNF-α, and IL-7. The method of claim 8, wherein the plurality of biomarkers consists of OPN, MCP-1/CCL2, MCP-3/CCL7, IL-17A, TNFRII, TNFRI, IL-4, IL-5, TNF-α, IL-7, IL-8/CXCL8, and MMP-9.

11. The method of claim 8, wherein the plurality of biomarkers consists of OPN, MCP-1/CCL2, MCP-3/CCL7, IL-17A, TNFRII, TNFRI, IL-4, IL-5, TNF-α, IL-7, Resistin, and IL-8/CXCL8.

12. The method of claim 8, wherein the plurality of biomarkers consists of OPN, MCP-1/CCL2, MCP-3/CCL7, IL-17A, TNFRII, TNFRI, IL-4, IL-5, TNF-α, IL-7, MMP-9, BLyS, and IL-8/CXCL8.

13. The method of claim 8, wherein the plurality of biomarkers consists of OPN, MCP-1/CCL2, MCP-3/CCL7, IL-17A, TNFRII, TNFRI, IL-4, IL-5, TNF-α, IL-7, BLyS, and IL-8/CXCL8.

14. The method of claim 1, wherein assessing the level of expression for each of the plurality of biomarkers comprises contacting at least a portion of the blood, serum, plasma, or saliva sample from the SLE patient with a kit configured to assess the level of expression for each of the plurality of biomarkers.

15. A kit comprising:
a set of reagents for determining expression levels of a plurality of biomarkers in a sample from a SLE patient, the plurality of biomarkers comprising Osteopontin (OPN), monocyte chemotactic protein-1 (MCP-1/CCL2), monocyte-specific chemokine 3 (MCP-3/CCL7), interleukin (IL-17A), tumor necrosis factor II (TNFRII), tumor necrosis factor receptor I (TNFRI), interleukin-4 (IL-4), interleukin-5 (IL-5), tumor necrosis factor-alpha (TNF-α), and interleukin-7 (IL-7).

16. The kit of claim 15, wherein the plurality of biomarkers consists of OPN, MCP-1/CCL2, MCP-3/CCL7, IL-17A, TNFRII, TNFRI, IL-4, IL-5, B lymphocyte stimulator (BLyS), TNF-α, and IL-7.

17. The kit of claim 15, wherein the plurality of biomarkers consists of OPN, MCP-1/CCL2, MCP-3/CCL7, IL-17A, TNFRII, TNFRI, IL-4, IL-5, TNF-α, IL-7, IL-8/CXCL8, and matrix metallopeptidase ((MMP-9).

18. The kit of claim 15, wherein the plurality of biomarkers consists of OPN, MCP-1/CCL2, MCP-3/CCL7, IL-17A, TNFRII, TNFRI, IL-4, IL-5, TNF-α, IL-7, Resistin, and interleukin-8 (IL-8/CXCL8).

19. The kit of claim 15, wherein the plurality of biomarkers consists of OPN, MCP-1/CCL2, MCP-3/CCL7, IL-17A, TNFRII, TNFRI, IL-4, IL-5, TNF-α, IL-7, matrix metallopeptidase ((MMP-9), B lymphocyte stimulator (BLyS), and interleukin-8 (IL-8/CXCL8). The kit of claim 15, wherein the plurality of biomarkers consists of OPN, MCP-1/CCL2, MCP-3/CCL7, IL-17A, TNFRII, TNFRI, IL-4, IL-5, TNF-α, IL-7, B lymphocyte stimulator (BLyS), and interleukin-8 (IL-8/CXCL8).
